(19) United States
(12) Patent Application Publication
(10) Pub. No.: US 2020/0055041 A1
(43) Pub. Date: Feb. 20, 2020

(54) DEVICE, SYSTEM AND METHODS FOR ELECTROPHYSIOLOGICAL INTERROGATION OF CELLS AND TISSUES

(71) Applicants: Nanosurface Biomedical, Inc., Seattle, WA (US); University of Washington, Seattle, WA (US)

(72) Inventors: Deok-Ho KIM, Seattle, WA (US); Alec S.T. SMITH, Seattle, WA (US); Eunpyo CHOI, Seattle, WA (US); Kevin GRAY, Seattle, WA (US)

(21) Appl. No.: 16/603,179

(22) PCT Filed: Apr. 6, 2018
(86) PCT No.: PCT/US2018/026534

§ 371 Date: Oct. 4, 2019

Related U.S. Application Data
(60) Provisional application No. 62/482,547, filed on Apr. 6, 2017.

(51) Int. Cl. B01L 3/00; G01N 33/483

(52) U.S. Cl. CPC B01L 3/50; B01L 2300/0645; B01L 2300/12; G01N 33/4836

(57) ABSTRACT

An apparatus for biological analysis includes a substrate, and an ion-permeable material with a textured surface on a first side of the ion-permeable material. The substrate is disposed proximate to a second side of the ion-permeable material, opposite the first side. A plurality of electrodes is disposed between the substrate and the ion-permeable material, where individual electrodes in the plurality of electrodes are positioned to measure an electrical signal that passes through the ion-permeable material.

### CROSS-REFERENCE TO RELATED APPLICATIONS

This application claims the benefit of U.S. Provisional Patent Application No. 62/482,547, filed Apr. 6, 2017, which is hereby incorporated by reference in its entirety.

### TECHNICAL FIELD

This disclosure relates generally to a testing device. In particular, examples of the present invention are related to devices to characterize electrical properties of cells and tissue.

### BACKGROUND

Cardiotoxicity is one of the primary reasons for drug failure, both during development and after market approval. Critically, due to a number of widely prescribed drugs being later withdrawn from the market because of unanticipated cardiac arrhythmogenic properties, the FDA now mandates that each new drug be tested for its potential to cause arrhythmias prior to use in humans. Animal models remain the gold standard for preclinical drug-toxicity detection. However, human cardiac physiology differs significantly from animals, leading to issues with false positive and false negative data, and poor preclinical predictions of compounds' effects at clinical trial. In addition, such in vivo assays are low throughput and expensive, increasing both the financial burden and time delay associated with these screening methods.

### BRIEF DESCRIPTION OF THE DRAWINGS

Non-limiting and non-exhaustive examples of the invention are described with reference to the following figures, wherein like reference numerals refer to like parts throughout the various views unless otherwise specified.

FIGS. 1A-1E illustrate an exemplary method for fabricating a conductive, ion-permeable, nanotopographic patterns onto a 48-well MEA plate, as well as the resultant electronic devices, according to one embodiment of the present disclosure.

FIGS. 2A-2H illustrates schematic details of a 384-well nanoMEA plate design according to one embodiment of the present disclosure.

FIGS. 3A-3C illustrate electrode layouts of a 384-well nanoMEA plate design, according to another embodiment of the present disclosure.

FIGS. 4A-4D depict an overview of a nanoMEA recording instrument system adapted for use with the nanoMEA plates, as well as additional electrode configurations, according to one embodiment of the present disclosure.

FIGS. 5A-5L show electrode sensitivity and baseline electrophysiological function of cultured cardiomyocytes on bare, PUA patterned, and Nafion patterned MEAs, according to one embodiment of the present disclosure.

FIGS. 6A-6I illustrate enhanced structural and functional properties of human cardiomyocytes on nanoMEAs, according to one embodiment of the present disclosure.

FIGS. 7A-7B illustrate slow skeletal troponin I protein expression from unpatterned and patterned hPSC-CM cultures, according to one embodiment of the present disclosure.

FIGS. 8A-8F illustrate effect of nanotopography on expression of oxidative stress markers in cultured hPSC-CMs, according to one embodiment of the present disclosure.

FIGS. 9A-9B show effects of nanotopography on spatial distribution of connexin 43 proteins in cultured hPSC-CMs, according to one embodiment of the present disclosure.

FIGS. 10A-10D illustrates baseline electrophysiology in flat and patterned hPSC-CM cultures, according to one embodiment of the present disclosure.

FIG. 11 illustrates a representative electrode, according to one embodiment of the present disclosure.

FIGS. 12A-12I illustrate electrophysiological response of flat and patterned hPSC-CMs to treatment with known arrhythmogenic compounds, according to one embodiment of the present disclosure.

FIGS. 13A-13D illustrate the response of flat and patterned hPSC-CMs to treatment with the conduction-blocking compound carbenoxolone, according to one embodiment of the present disclosure.

FIGS. 14A-14H illustrate structural and electrophysiological responses of normal and HCM hPSC-CMs to culture on nanoMEAs, according to one embodiment of the present disclosure.

FIG. 15 depicts 3D cardiac tissue responses to treatment with increasing doses of the hERG channel blocker cisapride, according to one embodiment of the present disclosure.

FIGS. 16A-16B depict an example method of device fabrication, and an exploded view of the complete device architecture, in accordance with an embodiment of the present disclosure.

FIGS. 17A-17C depict several electrode shapes, in accordance with an embodiment of the present disclosure.

FIG. 18 depicts a method of measuring the electrical properties of one or more cells, in accordance with the teachings of the present disclosure.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION

Examples of devices, systems, and methods for electrical characterization of cells and tissues are described herein. In the following description, numerous specific details are set forth to provide a thorough understanding of the examples. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one example” or “one embodiment” means that a particular feature, structure, or characteristic described in connection with the example is included in at least one example of the present invention. Thus, the appearances of the phrases “in one example” or “in one embodiment” in various places throughout this specification are not necessarily all referring to the same example. Furthermore, the particular features, structures, or characteristics may be combined in any suitable manner in one or more examples.

Drugs have been screened first using non-cardiac single-cell assays which may not fully recapitulate the biology of cardiomyocytes (CMs). This situation has spurred interest in screening with human pluripotent stem cell-derived cardiomyocytes (hPSC-CMs) as an alternative. While the use of simple hPSC-CMs represents an improvement over non-cardiac in vitro assays, these cells also have several major limitations that have inhibited widespread market adoption. First, hPSC-CMs are immature and typically possess a fetal phenotype, meaning they lack many important properties of adult human CMs and typically exhibit an inappropriate pattern of ion channel expression. Second, hPSC-CMs are primarily tested using a single-cell format. In addition to issues with variability, single cell analyses cannot address phenomena occurring at the multicellular level (e.g. drugs that interfere with cell-cell communication or many directly cytotoxic drugs). Even when cultured in a multicellular format, hPSC-CMs may not assume the highly aligned, anisotropic architecture of the intact myocardium. This means even high-density monolayers of hPSC-CMs may not accurately model electrophysiological behavior, which poses problems when attempting to model arrhythmia under the conditions of rapid, unidirectional electrical propagation that occurs within adult human heart tissue.

Nanotopographic substrates may be used to enhance the structural development of stem cell-derived cardiomyocytes. Such topographic stimuli are capable of altering the electrophysiological behavior of cultured cardiac monolayers. For example, nanopatterned culture plates that contain a substantially parallel (e.g., ±10° of rotation relative to parallel) array of nano-scale grooves and ridges may be used. It is appreciated that, in some embodiments, nanoscale may refer to structures from 1 nm to several microns in size. Nanotopographic surface structures, like the one described, promotes significant CM maturation and aligns the myocytes into a functional, electrically connected anisotropic monolayer. However, in order to investigate whether such substrate cues can improve the performance of cultured cardiac monolayers to a point where increased ability to predict drug efficacy and toxicity is possible, what is needed is an effective and reliable device, system and method for interrogating the cells cultured on such substrates for their electrophysiological characteristics. Such tools will provide a more accurate preclinical screening capability for drug-induced cardiotoxic risks by recapitulating the highly aligned architecture and functional phenotype of the mature human myocardium.

Preclinical assessment of drug-induced cardiotoxicity, and arrhythmogenic risk in particular, focuses on the study of changes in potassium currents (via analysis of hERG channel behavior) in non-cardiac cells coupled with animal studies of QT prolongation. Despite the success of this technique in identifying high-risk compounds, such screening methods may be too stringent, and some compounds that actually carry a minor risk are eliminated from further development. A lack of ability to model hERG-independent mechanisms, as well as issues with species-specific interactions, have further reduced confidence in the capacity for current preclinical screens to provide a high degree of accuracy in terms of predicting clinical outcomes. Accordingly, efforts have been undertaken to identify a more accurate preclinical cardiac screening paradigm, utilizing multiple ion channel studies, complementary in silico modeling, and analysis of human cardiomyocyte electrophysiological behavior using microelectrode arrays (MEAs). Human pluripotent stem cell-derived cardiomyocytes are important to MEA analysis methods as they remove reliance on animal cells and enable assessment of patient-specific electrophysiological performance within human myocardial model. However, these cells are typically analyzed at a fetal stage of development, which limits their ability to predict more mature cardiac behavior accurately. Multiple methods for enhancing cardiac maturation in vitro have been described in the prior art, and analysis of such engineered constructs has demonstrated a capacity for matured cardiac tissues to elicit responses to drug treatment that more closely recapitulate that of the native human myocardium. Unfortunately, many of the developed methods utilize unique platforms with configurations and/or analysis methods that can present difficulties when attempting to scale up to high-throughput formats. Thus, a device, system and method to improve hPSC-CM development that can be readily adapted to existing high-throughput screening methodologies, such as MEA, are highly desirable.

What is needed, therefore, is a device, system and method for cell assays that improves structural organization and maturation of the cultured cells, such as hPSC-CM monolayers, while allowing more accurate investigation of electrophysiological functions of such cultured cells in a high-throughput manner. A nanotopographically-patterned microelectrode array (nanoMEA) device in accordance with the teachings of the present disclosure constitutes a simple and reproducible method that utilizes bioinspired matrix nanotopographic cues to drive cardiac phenotypic development in vitro. Experimental data obtained with the nanoMEA system demonstrate the impact these phenotypic changes have on both baseline and drug-induced responses of human cardiomyocytes to treatment with known cardiotoxic compounds. Specifically, the nanoMEA assay of the present disclosure can identify the enhanced effect of compounds that target structurally-defined elements within the cardiac cell and demonstrate the capacity for patterned surfaces to exacerbate cardiomyopathic behavior in diseased cells. The nanoMEA system of the present disclosure represents the means to conduct more in-depth analysis of how structural development and organization within cardiac cells and tissues affect functional output, thereby enhancing the capacity for preclinical MEA-based screens to predict drug efficacy and toxicity in humans.

As stated, embodiments discussed below are directed to microelectrode array (MEA) devices, systems and methods that can be used for high-throughput electrophysiology assays for drug screening applications and disease studies. More particularly, the present disclosure is directed to an electrophysiology assay system equipped with a nanotopographically-patterned culture plate integrated with MEAs.

### Integration of Nanotopographically-Patterned, Ion-Permeable Thin Films Into Microelectrode Arrays

Experimental results show that a nanotopography configuration of the present disclosure has a significant impact on the electrophysiological function of hPSC-CM monolayers. One objective of the present disclosure is to develop a more accurate and reliable method for interrogating the cells cultured in such nanotopographically-patterned medium for changes in field potentials and propagation patterns.

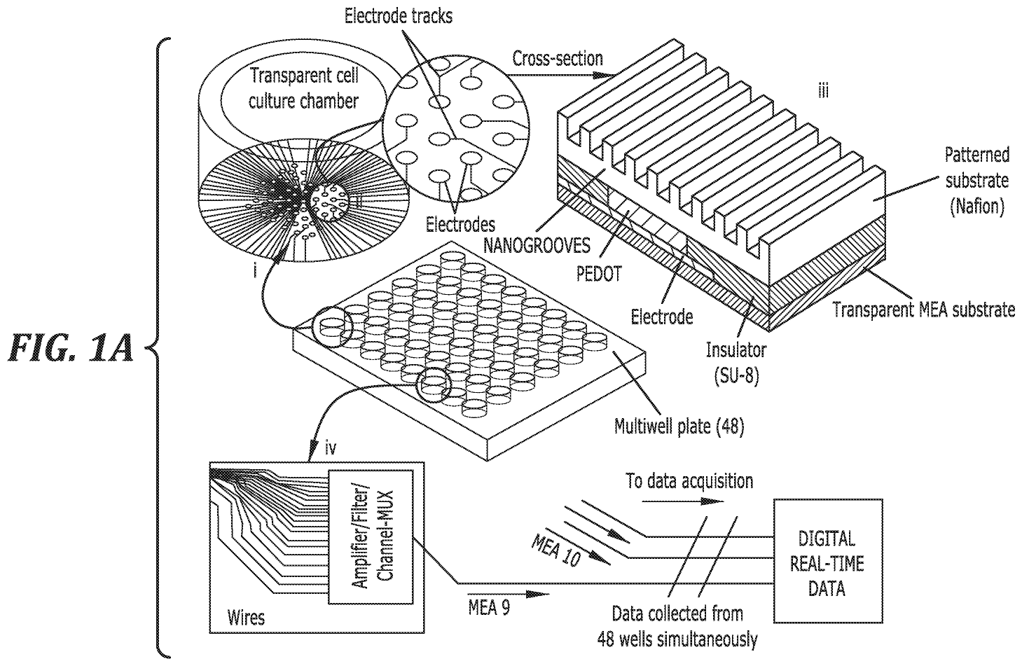

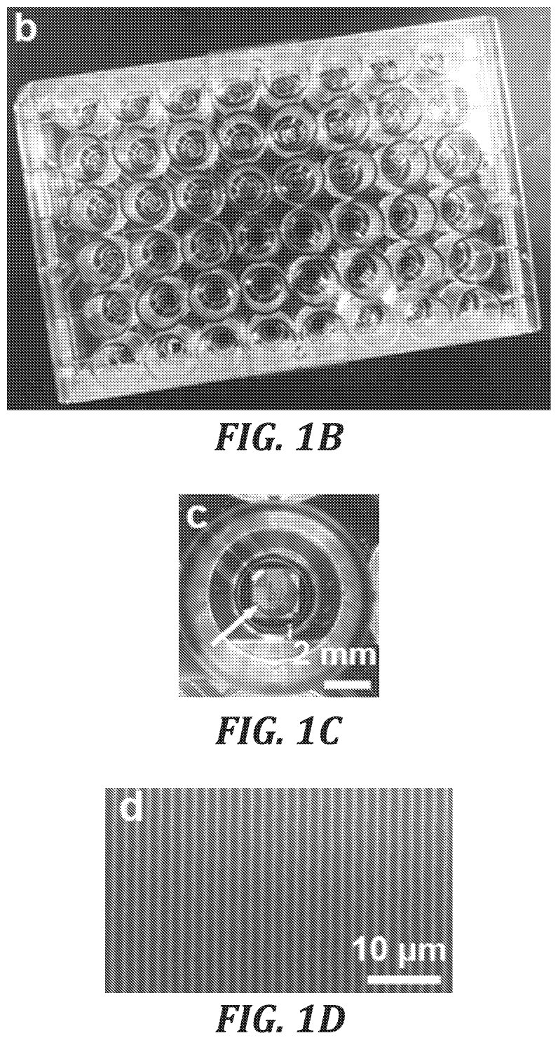

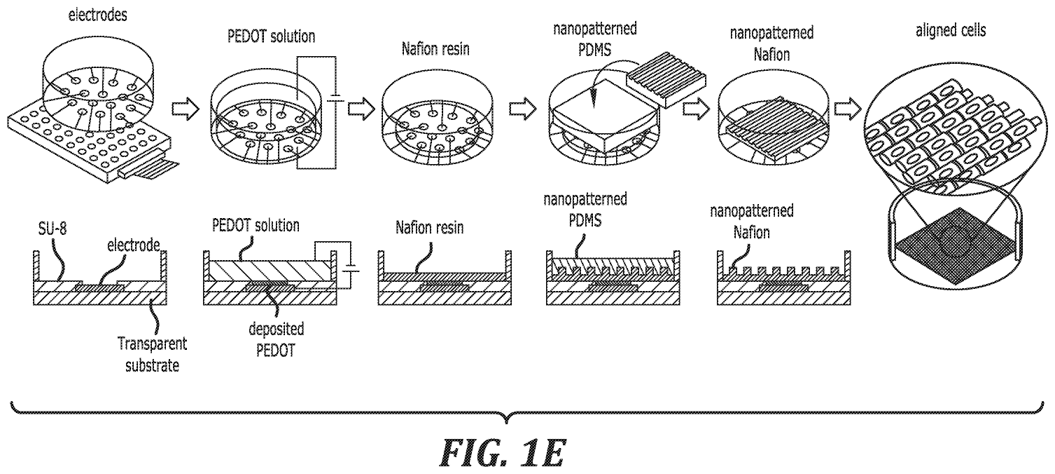

According to an embodiment of the disclosure as shown in FIG. 1A-1E, each well of the 48-well MEA plate is configured to support independent cell cultures for high-throughput analysis. Within each well, the electrode bed facilitates recording of field potentials generated by the overlying cells. Nanotopographic patterns of ion-permeable material such as Nafion is applied to each well (or a subset of wells) to promote cellular alignment and functional development. In some embodiments, the ion-permeable material comprises Nafion, such as commercially available products (e.g., Sigma Aldrich, NafionR perfluorinated resin solution (Cat. Nos. (5%) 510211-100ML and (20%) 663492-25ML). Unless specifically indicated otherwise, the term “Nafion” as generally used can refer to the Nafion or a composition containing Nafion polymer at a concentration sufficient to permit ion permeability in the final product. As the ion-permeable nature of the nanotopographically-patterned substrate enables efficient signal detection from overlying cells, captured signals are relayed, via an amplifier, to a software program for subsequent analysis. Given its ionic properties and superior thermal and mechanical stability, in one embodiment, Nafion is the preferred material for forming the nanotopographically-patterned substrate as it enables reliable signal capture of cardiomyocyte field potentials from underlying electrodes, while retaining the rigidity required to form high-fidelity nanoscale 3D topographic structures. However, other nanoporous and/or ion-permeable materials such as polyethylene terephthalate (PETG), track-etched membranes, gelatin, Matrigel, any pattern-able hydrogel, poly-acrylamide, N-isopropylacrylamide (poly-NIPAM), agarose gels, and/or dextran gels may be used instead of Nafion or in combination with Nafion or one or more of the above-described materials.

In one embodiment, as shown in FIGS. 1B-1D, conductive ionomeric nanopatterns are fabricated from a nanoporous polymer (Nafion) onto each well of a commercially available, 48-well microelectrode (MEA) array plate from Axion Biosystems to promote hPSC-CM maturation while simultaneously enabling high-throughput assessment of patterned cardiomyocyte function. FIG. 1C shows low magnification image of nanotopography applied to a single well of the 48-well MEA plate. The presence of the nanoscale features causes light diffraction on the surface, giving the patterns a green-orange color. Pattern fidelity is assessed by scanning electron microscopy (SEM) as shown in FIG. 1D.

FIG. 1E illustrates the process for forming nanotopographic patterns from the Nafion resin. A parallel array of grooves and ridges having widths of approximately 800 nm promotes optimal structural development in hPSC-CMs, in terms of cell area, elongation, and sarcomere length (accordingly, structures that perform one or more of these functions along with other functions may be considered shaped to receive cells). Accordingly, the Nafion nanopatterns in one embodiment of the present disclosure may be formed in grooves and ridges spanning a width of approximately 800 nm in their respective width. However, the groves and ridges can have other widths (e.g, 400 nm, 500 nm, 600 nm, 700 nm, or the like). One of skill in the art will appreciate there are many structures that are shaped to receive cells. Each well within the 48-well plate in FIG. 1E contains a separate, transparent MEA substrate. Pristine wells are first treated with PEDOT to improve the sensitivity of the base electrode. A drop of Nafion resin is then applied to the substrate, and a PDMS mold is pressed into it. After overnight curing, the PDMS mold is removed to reveal Nafion topographic substrates underneath.

Although the embodiment of the present disclosure shown in accordance with FIGS. 1A-1E provides a nanoMEA device that exhibits positive effects that nanotopographically-patterned surfaces have on cardiomyocyte function and drug responses, these MEA well layouts, in some embodiments, may suffer from certain suboptimal design features that limit the ability to fully capitalize on the advantages offered by nanotopography of the culture plate. These include limitations on the plating area, which leads to crowding of the cells over the electrodes and reduces capacity for cells to respond to the underlying topographic cues. The 48-well MEA design disclosed in FIGS. 1A may require cells to be plated in small, 6-8 μL droplets in order to prevent cells covering ground electrodes. This drop-seeding procedure leads to well-to-well variability in terms of plating density and cell spacing based on the degree of spread that the droplet undergoes. When applied onto the nanoMEA plates, this variability means cell alignment and maturation can vary based on specific cell density and degree of crowding. In order to capitalize on the advantages provided by the nanotography of the substrate, and to improve culture reproducibility, an improved design that enables more consistent plating conditions for cell seeding is also described herein.

The number of electrodes per well may also limits the overall throughput of the system. A new electrode layout that allows a reduction in electrode numbers per well may enable sufficient data collection with significantly greater throughput (for example, 384-wells) while, at the same time, maintaining the same total electrode numbers. A nanoMEA plate that incorporates both an optimized well design that facilitates reliable plating of cardiac monolayers and an improved electrode layout that allows longitudinal propagation analysis with increased throughput is thus highly desirable.

It should be noted that the current drop-seeding protocol for conventional MEA plates may be suboptimal for high-capacity multi-well formats such as 384-well plate due to the need to avoid plating cells on grounding electrodes within much smaller wells. Furthermore, high cell densities over the microelectrodes can produce suboptimal alignment in response to the underlying nanotopography. Thus, a MEA culture plate that allows seeding of the cells over a precisely defined area is further desirable.

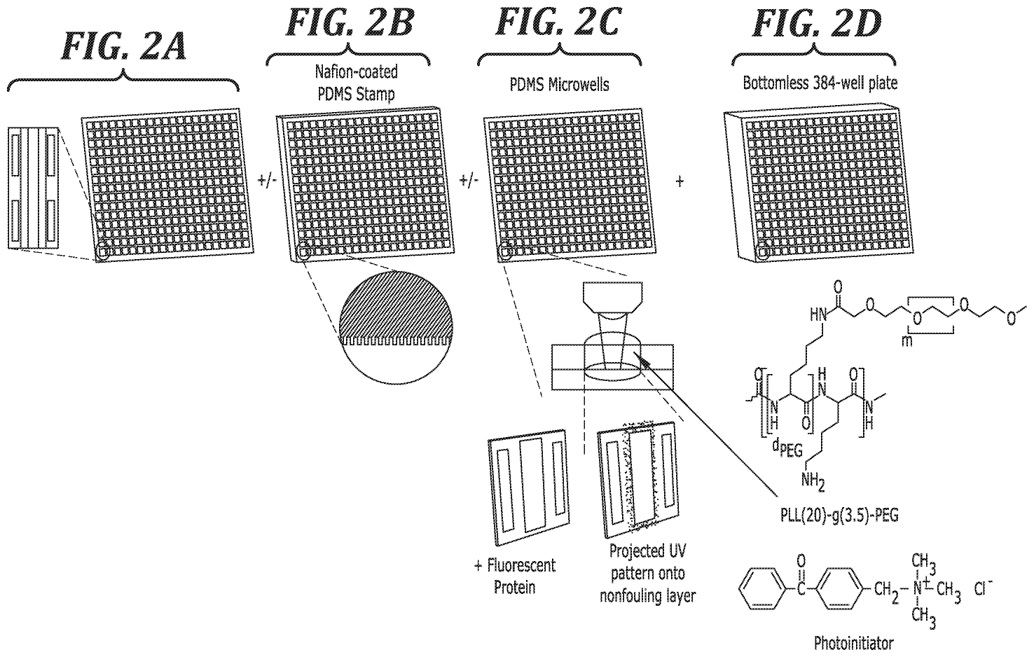

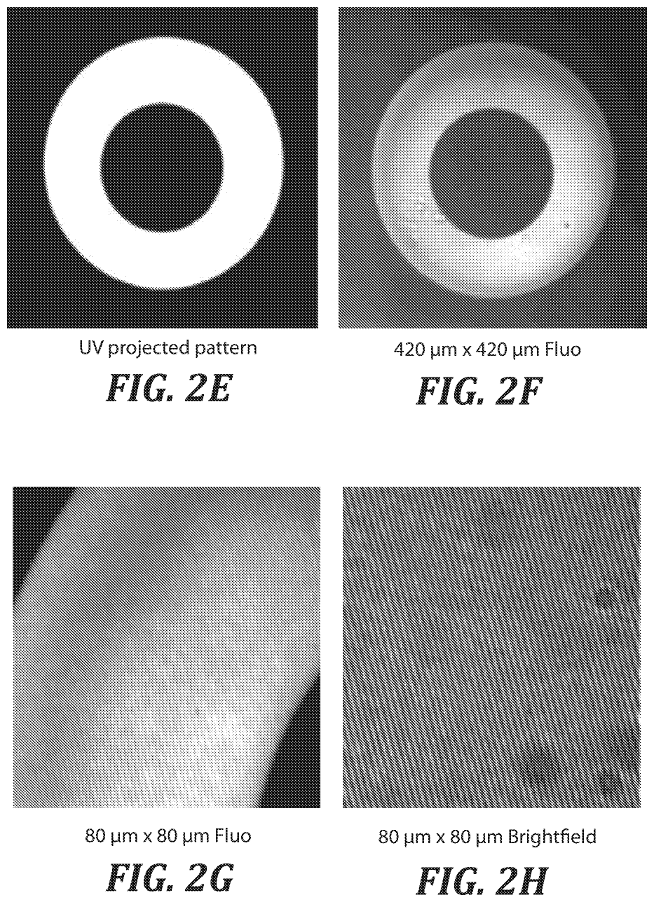

FIGS. 2A-2H illustrate schematic details of a 384-well nanoMEA plate design according to another embodiment of the present disclosure. Two circular electrodes are for stimulation and field potential recording and large rectangular electrodes serve as grounds. Once electrodes are deposited on a MEA substrate, a Nafion-coated PDMS stamp will be applied to the MEA substrate (FIG. 2B). After curing, removal of PDMS will reveal Nafion nanostructures coating the electrodes. A second PDMS stamp is then applied to the surface to create microwells that restrict application of subsequent surface treatments to specific areas of the well (FIG. 2C). UV light and a crosslinking agent is then used in conjunction with a microscope-based projection system to project the desired pattern upon each well-bottom. Once patterned, the PDMS wells are removed and each well is fluidically isolated by application of 384 bottomless well plates to the MEA substrate (FIG. 2D). UV pattern projection onto nanopatterned surfaces creates areas that are permissive to protein binding and other areas that are restrictive (FIGS. 2E-2H). In this manner, an entire well can be coated with fibronectin and yet protein binding controlled, which in turn restricts cell attachment.

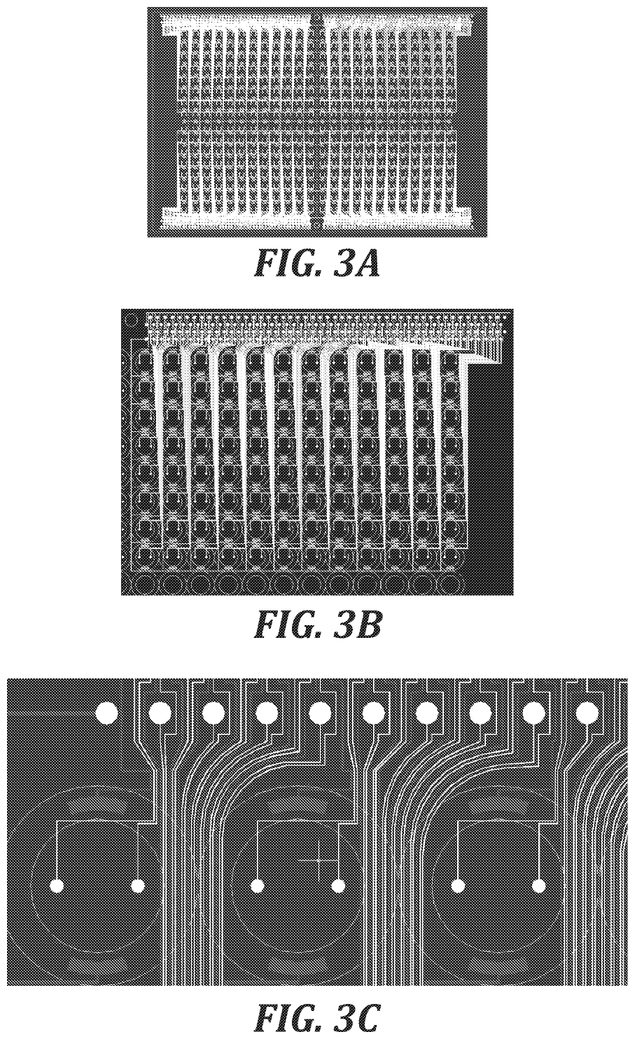

In another embodiment of the present disclosure, the number of electrodes per well is reduced to the bare minimum required for cardiac applications in order to facilitate assessment of nanotopographically-influenced conduction velocities while simultaneously increasing throughput to a 384-well format. In an exemplary design, 2×100 μm diameter microelectrodes are positioned at the distant ends of rectangular 0.5 mm×3 mm seeding area (FIG. 3C). For conduction velocity measurements, the culture is paced at one end to localize the epicenter of activation, but is otherwise evaluated during spontaneous contraction. The anisotropy of spreading resistance, implemented through large grounding electrodes closely situated to the pacing electrode coupled with the substantial distance between pacing and recording electrodes biases any artifact currents toward the grounding electrodes and not the recording electrodes, minimizing discharge artifacts. The substantial distance between pacing and recording electrodes also minimize the coincidence of the initial biphasic stimulus with the recorded field potential waveform. Such electrode layout allows for effective conduction velocity measurement and QT interval evaluation without the need for additional signal-processing. This 2-electrode configuration allows relatively simple scaling to 384-wells.

FIGS. 3A-3C depict exemplary layouts of a 384-well MEA, according to another embodiment of the present disclosure. FIG. 3A shows 384-well MEA deposited on 8-inch glass wafer. FIG. 3B shows a magnified image of a portion of such 384-well MEA. FIG. 3C shows a magnified image of the layouts of recording electrodes having a line width of 15 um and diameter of 300 um and ground electrodes having a line width of 30 um and size of approximately 1400 um×300 um.

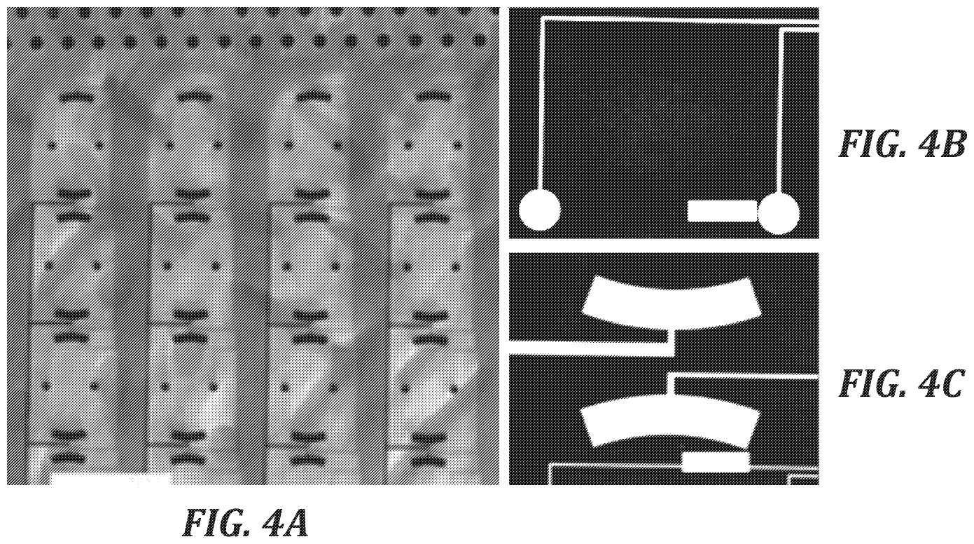

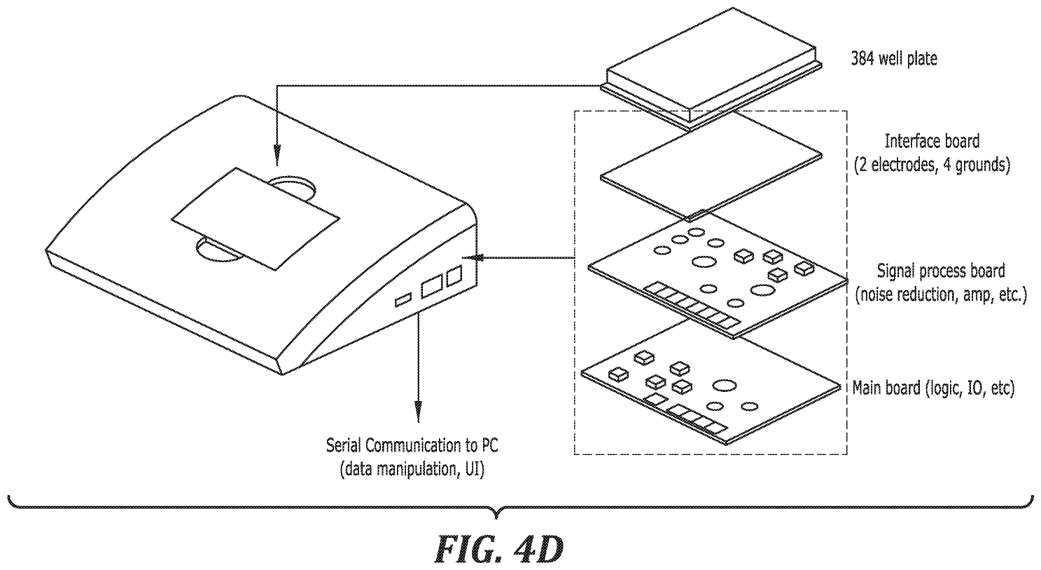

Additionally, nanoMEA recording hardware and accompanying software can be configured to utilize such novel 384-well plate design for high-throughput assessment of drug-induced arrhythmogenic potential. Such nanoMEA recording instrument comprises hardware, firmware (i.e., embodiments of “logic”) and are shown in FIG. 4D. FIG. 4A shows an exemplary multiwell layout of electrodes. FIG. 4B and FIG. 4C show an exemplary layout of two recording electrodes and two ground electrodes for each well respectively. In this layout, one electrode is used to pace the cells and a second electrode is used to record field potential waveforms as well as conduction velocity between the pacing and recording electrodes. The other two electrodes serve as grounds for both the pacing (e.g., applying a voltage) and recording electrodes. In one embodiment, the ground electrodes are designed to be significantly larger than the pacing and recording electrodes, allowing bath-seeding of each well as opposed to inherently inconsistent drop-seeding approaches, thereby achieving optimal cell alignment and well-to-well consistency. FIG. 4D depicts schematic details of the organization of the recording instrument for analyzing cardiac field potential data. In some embodiments, a user interface on a computer is used to control data acquisition of the system depicted. Elements of the hardware include an interface board (IB), a signal process board (PB) and a main board (MB). The interface board constitutes a mating board for directly connecting the consumable 384-well nanoMEA plate. The signal process board is designed to maximize the signal-to-noise ratio, as well as amplification of the detected output from the interface board. The hardware may include temperature and atmospheric controllers to provide stable conditions to cells while recording electrical or electrophysiological activity of the cells. Also, stimulation functionality may be incorporated into the hardware such that stimulation settings for each well can be dictated independently. The user interface is run by a computer, which is serially connected to the hardware. The software is written to enable the user interface to display multi-channel simultaneous outputs from the 384-wells. In addition, the software provides a variety of noise filters to clean recorded signals, as well as functions for depolarizing peak identification, conduction speed calculation, field potential duration, and beat-regularity measurement. Pre-selected and automated protocols, recipes, and reporting function could be programmed to facilitate reproducibility and throughput. Such software may be written in MATLAB and enable export to analysis software in ASCII and delimited text formats.

The nanoMEA plate/electrode designs disclosed above enable even cell distribution and the formation of robust monolayers with highly aligned morphologies while keeping ground electrode regions clean. Such improved well design and monolayer generation can deliver significant improvements in markers of cardiac maturation by western blot and immunocytochemistry. Cell densities that enable tight monolayer formation without promoting the layering of cells on top of each other will promote the greatest levels of maturation. The software, as optimized for the 384-well nanoMEA, can provide simultaneous recording from all 384-wells with accurate assessment of field potential and conduction velocities relative to the nanopattern direction. It is expected that such multiwell nanoMEA designs will promote further improvements in hPSC-CM electrophysiology maturation metrics over results obtained with conventional MEA systems, making more accurate and more predictive high-throughput assessment of drug-induced arrhythmogenic potential.

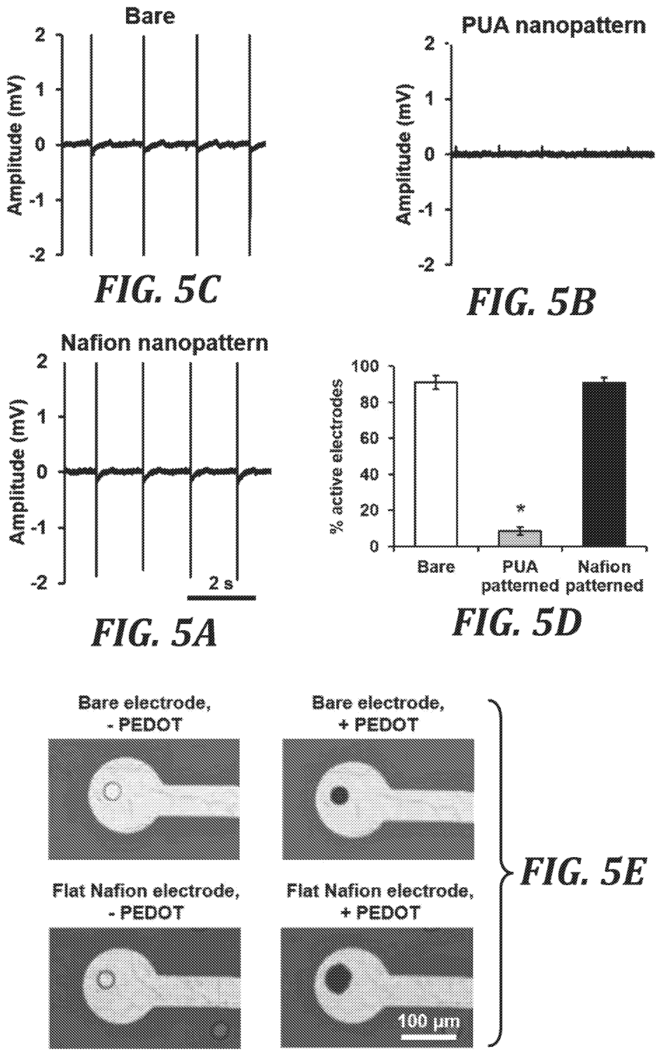

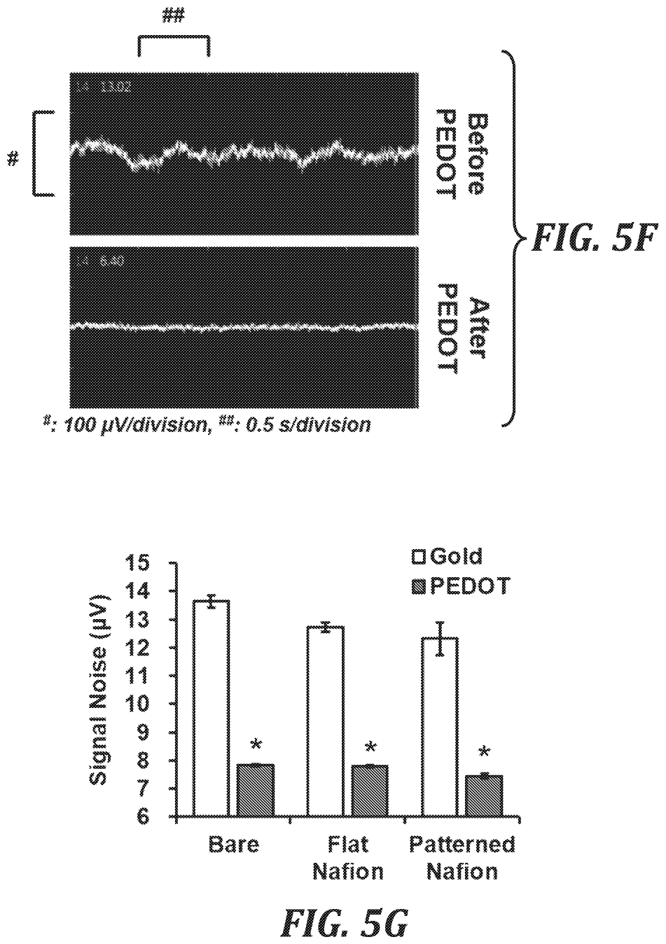

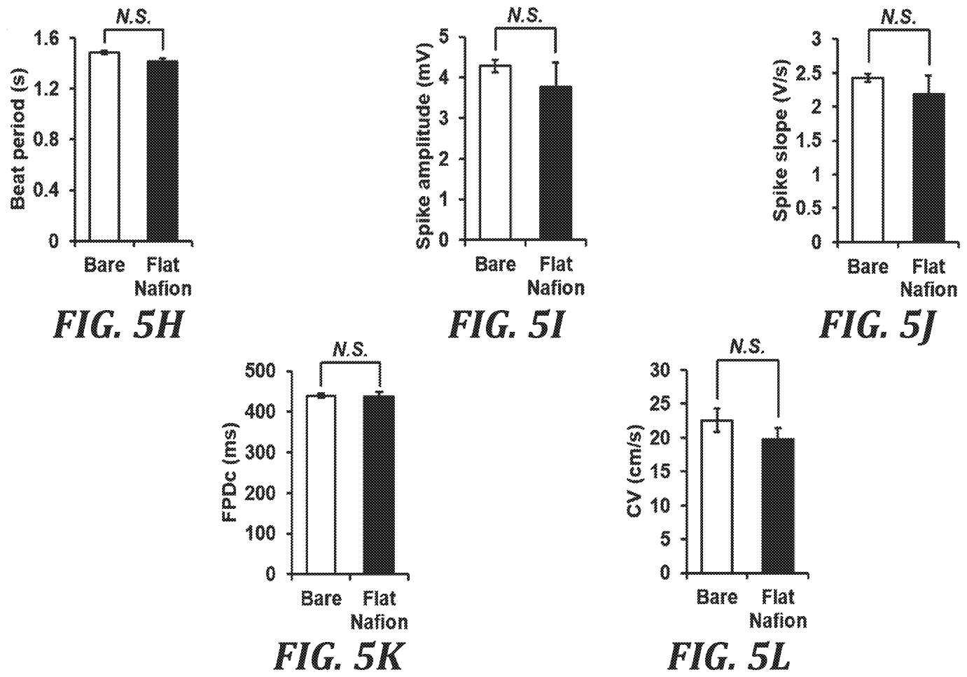

To evaluate whether application of Nafion substrates to MEAs has an impact on signal acquisition, impedance measurements and electrode noise on uncoated MEAs, MEAs coated with a flat Nafion layer, and MEAs coated with nanotopographically-patterned Nafion were carried out (see FIGS. 5A-5L). FIGS. 5A, 5B and 5C show field potential recording from hPSC-CMs on bare MEAs, PUA nanoMEAs, and Nafion nanoMEAs, respectively. FIG. 5D shows percentage of MEA electrodes from which hPSC-CM signal detection could be clearly distinguished above background noise. In addition, recordings were analyzed from electrodes that had been treated with the conductive polymer, Poly(3,4-ethylenedioxythiophene) (PEDOT), prior to Nafion application and those that had not, as shown in FIG. 5E. PEDOT is used to increase the sensitivity of electrodes and was investigated as a means to improve signal capture from the microelectrodes. FIG. 5F shows representative noise recordings from multiwell MEAs made in saline solution demonstrating the reduction in noise due to electrode coating with PEDOT. FIG. 5G shows quantification of noise recorded from bare, flat Nafion, and nanopatterned (NP) Nafion electrodes with and without PEDOT treatment. In all conditions examined, PEDOT application was found to significantly reduce electrode noise and impedance readings, but no difference between uncoated and Nafion coated electrodes was observed. Without PEDOT, electrode noise measurements across 32 independent electrodes were recorded as 13.64 μV±0.23, 12.73 μV±0.17, and 12.33 μV±0.58 for uncoated, flat Nafion, and nanotopographically-patterned Nafion electrodes, respectively. The presence of PEDOT reduced these readings to 7.84 μV±0.04, 7.79 μV±0.04, and 7.45 μV±0.05, respectively. Similarly, impedance magnitude measurements for uncoated (bare), flat Nafion, and nanotopographically-patterned Nafion electrodes went from 2.56±0.58, 1.57±0.16, and 2.41±0.20 MΩ, respectively, to 0.013±0.00027, 0.010±0.00054, and 0.015±0.0019 MΩ following PEDOT addition, a decrease in impedance of over 100-fold.

To evaluate the impact of Nafion coating on signal detection further, electrophysiological recordings were collected from hPSC-CM monolayers at day 7 using uncoated and flat Nafion coated electrodes. FIGS. 5H-5L show differences in measurement in beat period (FIG. 5H), depolarizing spike amplitude (FIG. 5I), depolarizing spike slope (FIG. 5J), field potential duration corrected for beat period (FPDc) (FIG. 5K), and conduction velocity (CV) (FIG. 5L). In all presented data, *p<0.05. No significant differences in baseline electrophysiological metrics were detected between cells maintained on bare MEAs and those coated with Nafion. As such, the presence of a Nafion layer between the cells and underlying electrodes was determined to have a negligible impact on the function and physiological performance of hPSC-CMs and the signal capture capabilities of the nanoMEA device.

### Nanotopographically-Patterned Nafion Substrates Promote Structural and Functional Maturation of Stem Cell-Derived Cardiomyocytes

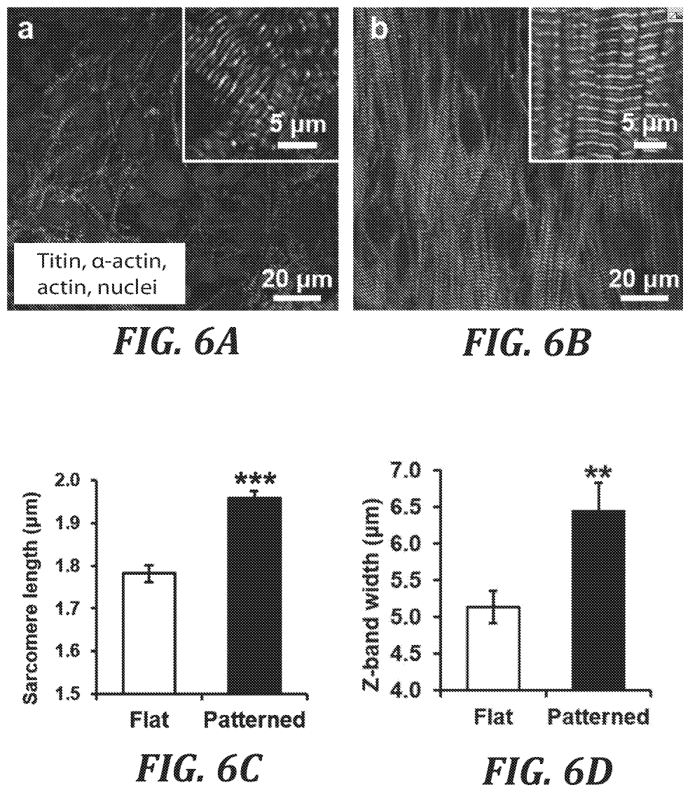

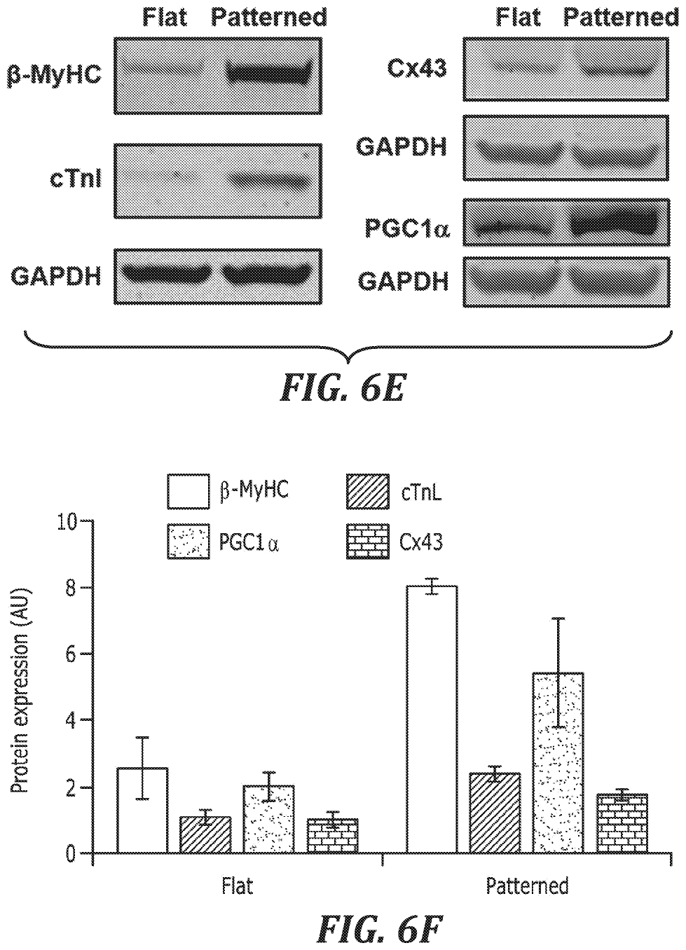

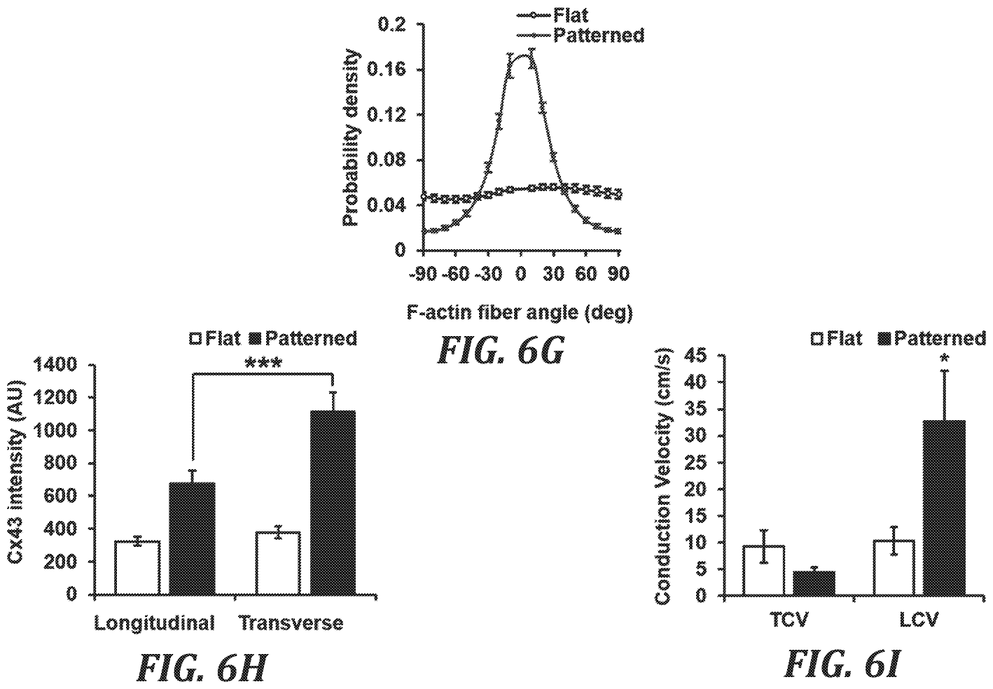

Additionally, to demonstrate that nanotopographically-patterned Nafion substrates are capable of promoting structural maturation of hPSC-CM monolayers to a similar degree as seen previously with single isolated hPSC-CMs and neonatal rat ventricular myocyte monolayers, immunocytochemical analysis was used to assess cell morphology and sarcomere structure in unpatterned and patterned hPSC-CM cultures. FIG. 6A shows an immunostained image of hPSC-CMs on flat Nafion substrates while FIG. 6B shows an immunostained image of hPSC-CMs on nanotopographically-patterned Nafion substrates, with each inset in the figure showing detail of the sarcomeric structures present therein, clearly demonstrating how Nafion nanotopography alters hPSC-CM structural development. The cells on flat substrates in FIG. 6A exhibit random orientations with poorly organized sarcomeres, whereas nanotopographically-patterned cells in FIG. 6B displays anisotropic morphologies with ordered myofibrils and regular z-band alignment. Both sarcomere lengths and z-band widths were found to increase on nanotopographically-patterned substrates, as shown in FIG. 6C and FIG. 6D; an accepted trait for cardiomyocyte maturation. Specifically, sarcomere length in cells maintained on flat surfaces was found to average 1.78 μm±0.02 (n=99), which increased to 1.96 μm±0.02 (n=100) in nanotopographically-patterned cells. Similarly, z-band widths in cultured cardiomyocytes were measured at 5.14 μm±0.21 (n=110) and 6.44 μm±0.39 (n=100) on flat and patterned surfaces, respectively.

In addition, quantification of F-actin alignment in unpatterned and patterned cells, as shown in FIG. 6G, clearly illustrates the polar nature and anisotropic cytoskeletal organization of cardiomyocytes maintained on nanotopographically-patterned substrates. FIG. 6G is a histogram detailing frequency of F-actin fibers in cultured cardiomyocytes that possess a given angle of alignment relative to vertical. All images collected from patterned cultures were oriented so that the pattern ran vertically.

Immunoblot analysis of protein lysates collected from unpatterned and patterned hPSC-CMs shown in FIG. 6E and FIG. 6F highlights the fact that nanotopographic substrates promote an upregulation of β-myosin heavy chain (β-MyHC), cardiac troponin I (cTnI), connexin 43 (Cx43), and peroxisome proliferator-activated receptor gamma coactivator 1-α (PGC1-α). FIG. 6E depicts immunoblot results from unpatterned and patterned hPSC-CMs, detailing expression levels of β-MyHC, cTnI, Cx43, and PGC1α, as well as GAPDH internal controls. FIG. 6F depicts densitometric analysis of band intensity, providing quantification of the changes in protein expression illustrated in FIG. 6E.

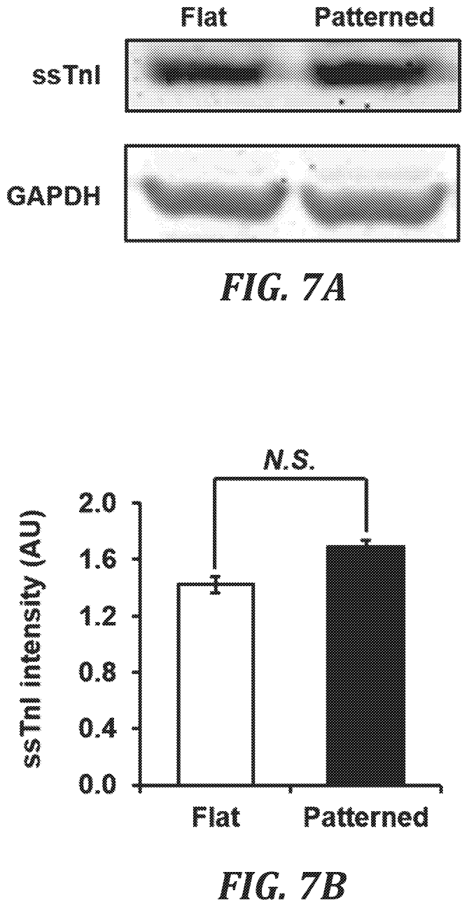

However, no significant difference was observed in levels of slow skeletal troponin I (ssTnI) expressed in cells maintained on flat versus patterned substrates, as shown in FIGS. 7A-7B. FIG. 7A depicts immunoblot blot results illustrating expression of ssTnI, as well as GAPDH internal control, in cardiomyocytes maintained on flat and patterned surfaces. FIG. 7B depicts densitometric analysis of ssTnI western blot results (n=2). Data is normalized to GAPDH and expressed in arbitrary units (A.U.). No significant difference was observed between conditions (p=0.33).

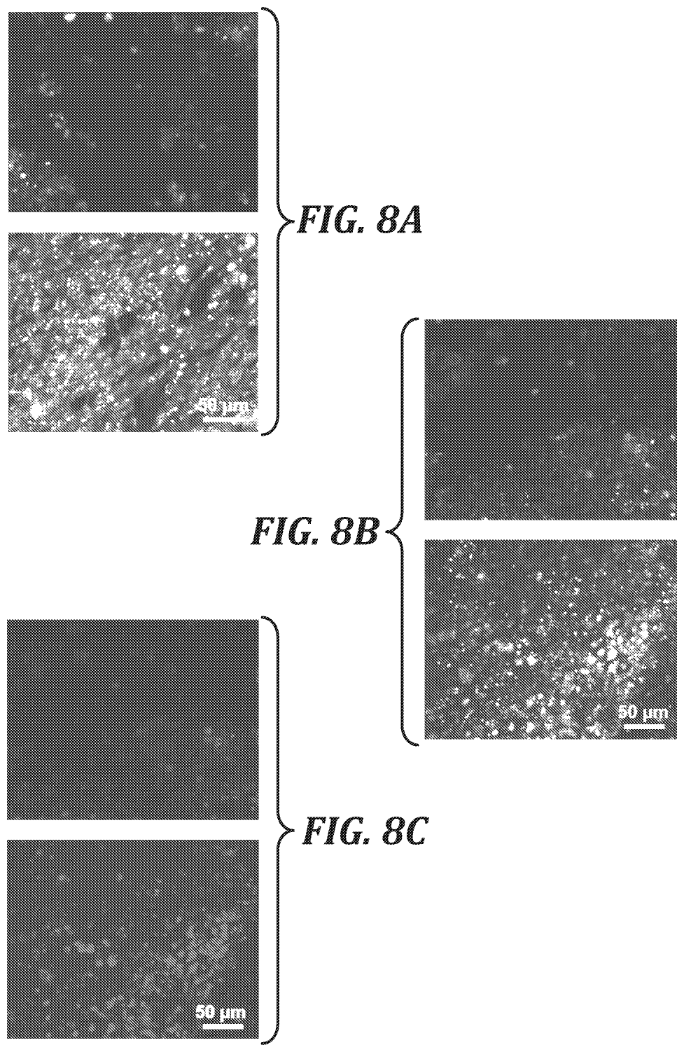

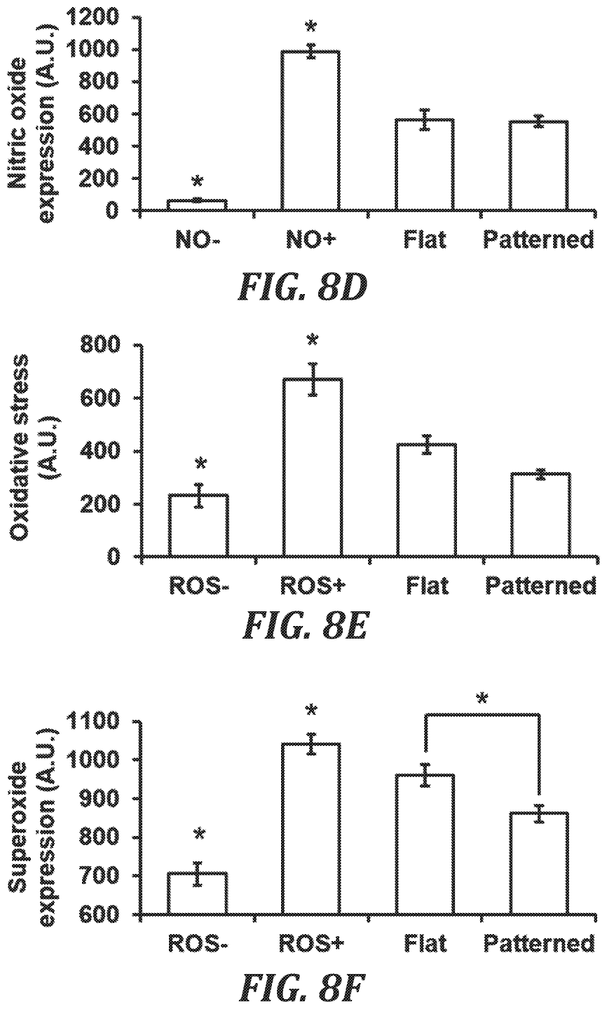

PGC1-α is known to perform a central controlling role in global oxidative metabolism through induction of mitochondrial biogenesis and tuning of the intrinsic metabolic properties of mitochondria_ENREF_35. Furthermore, PGC1-α has also been shown to act as a key regulator of reactive oxygen species (ROS) removal via the expression of several ROS-detoxifying enzymes, including glutathione peroxidase 1 (GPx1) and superoxide dismutase 2 (SOD2). Based on this understanding, a fluorometric cellular ROS detection assay was performed on unpatterned and patterned hPSC-CMs to determine whether upregulation of the PGC1-α protein in patterned cells had any notable impact on oxidative stress. FIGS. 8A-8F show the effect of nanotopography on expression of oxidative stress markers in cultured hPSC-CMs. FIG. 8A shows a representative image from patterned cardiomyocyte culture stained with a nitric oxide (NO) detection reagent (red) following treatment with an NO scavenger (c-PTIO; top) and an NO inducer (L-arginine; bottom). FIG. 8B shows a representative image from patterned cardiomyocyte culture stained with an oxidative stress detection reagent (green) following treatment with a reactive oxygen species (ROS) inhibitor (N-acetyl-L-cysteine; top) and an ROS inducer (Pyocyanin; bottom). FIG. 8C shows a representative image from patterned cardiomyocyte culture stained with a superoxide detection reagent (blue) following treatment with an ROS inhibitor (top) and an ROS inducer (bottom). FIG. 8D shows quantification of pixel intensity in images collected from cardiac cultures stained with an NO detection reagent. Samples analyzed were patterned cells treated with an NO scavenger (NO−), patterned cells treated with an NO inducer (NO+), untreated unpatterned cells, and untreated patterned cells. NO− was significantly lower than all other groups, while NO+ was significantly higher than all other groups examined. No difference was observed between flat and patterned samples. FIG. 8E shows quantification of pixel intensity in images collected from cardiac cultures stained with an oxidative stress detection reagent. Samples analyzed were patterned cells treated with an ROS inhibitor (ROS−), patterned cells treated with an ROS inducer (ROS+), untreated unpatterned cells, and untreated patterned cells. ROS+ exhibited significantly higher values then all other groups, while ROS− was significantly lower than all other groups examined, with the exception of the patterned samples. No significant difference was observed between flat and patterned samples. FIG. 8F shows quantification of pixel intensity in images collected from cardiac cultures stained with a superoxide detection reagent. Samples analyzed were patterned cells treated with an ROS inhibitor (ROS−), patterned cells treated with an ROS inducer (ROS+), untreated unpatterned cells, and untreated patterned cells. Superoxide presence was significantly lower than all other groups in the ROS− samples, while it was significantly higher than all other groups examined in the ROS+ samples. A significant difference was also observed between flat and patterned samples. In all presented data, *p<0.05.

Overall, quantification of nitric oxide and oxidative stress stains showed no difference between unpatterned and patterned samples, although a trend towards lower levels in patterned cultures was observed for the latter. Patterned cardiomyocytes did exhibit a significant reduction in superoxide expression, compared with unpatterned controls, providing support for the hypothesis that nanotopography enhances the oxidative metabolic capacity of hPSC-CMs. In addition, the collected data demonstrate that nanotopographically-patterned culture surfaces exert little effect on the stress state of cardiomyocytes maintained on these surfaces. Thus it is appreciated that any differences observed in electrophysiological function between unpatterned and patterned cells are due to the maturation state of the cells and not a by-product of a stress response in patterned cultures.

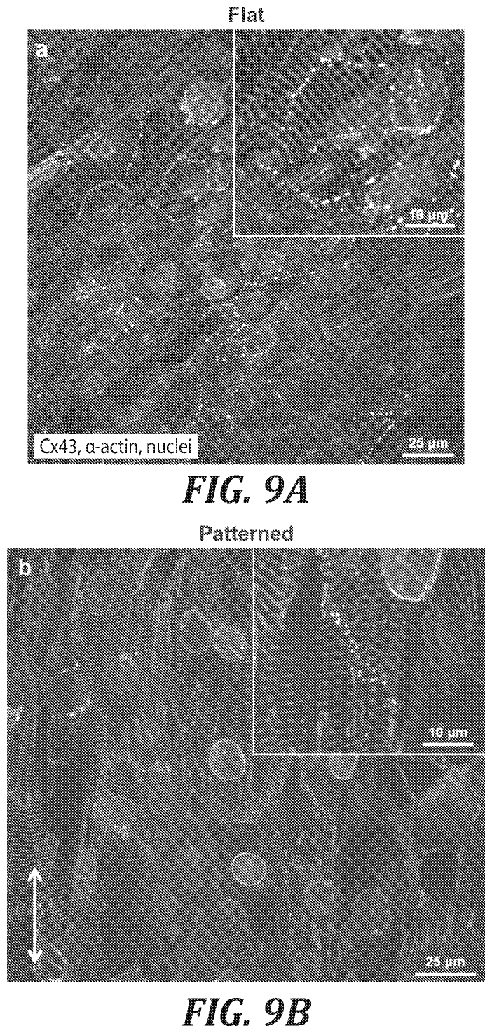

In addition to immunoblot data revealing an upregulation in Cx43 expression in patterned cells, analysis of Cx43 localization in immunostained cultures revealed that Cx43 was more highly expressed in the transverse orientation (relative to the underlying nanotopographic patterns), suggesting that gap junction accumulation was occurring at the polar ends of patterned cells (FIG. 6H and FIGS. 9A-9B). FIG. 6G depicts quantification of pixel intensity in images collected from hPSC-CM cultures stained with a primary antibody that targets Cx43. FIG. 9A-9B show the effect of nanotopography on spatial distribution of Cx43 proteins in cultured hPSC-CMs. FIG. 9A shows a representative immunostained image of hPSC-CMs on flat Nafion substrates showing random distribution of gap junctions. FIG. 9B shows a representative immunostained image of hPSC-CMs on patterned Nafion substrates showing more polar orientation of gap junctions. However, no directional preference for Cx43 expression was observed in unpatterned cells, further highlighting the impact of nanotopography on subcellular organization in hPSC-CMs.

Overall, the anisotropic nature of the cultured cells, the improvements in sarcomere length, myofibril alignment, and gap junction protein expression and localization, as well as the significant upregulation of cardiac specific contractile machinery and metabolic regulator proteins, all serve to underscore the ability for Nafion nanotopographic patterns to enhance the phenotypic development of cultured hPSC-CMs. These results support and build upon those published previously using alternative polymeric materials and confirm the ability for Nafion nanotopographic patterns to promote the establishment of more physiologically relevant cardiac cell sheets for use in downstream applications.

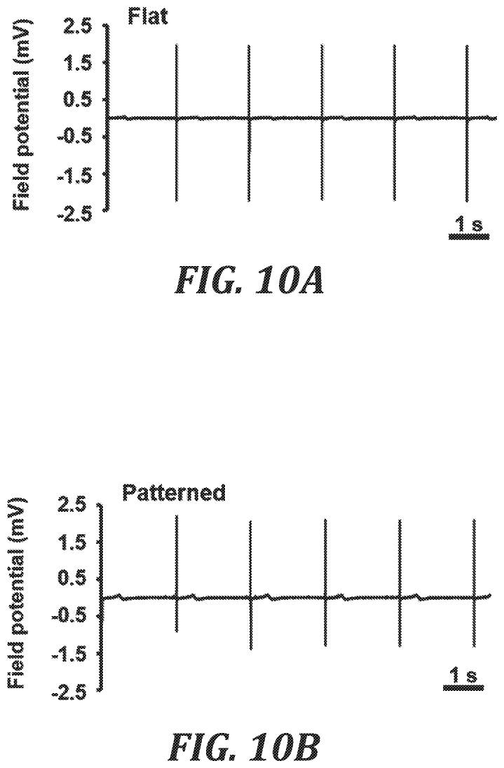

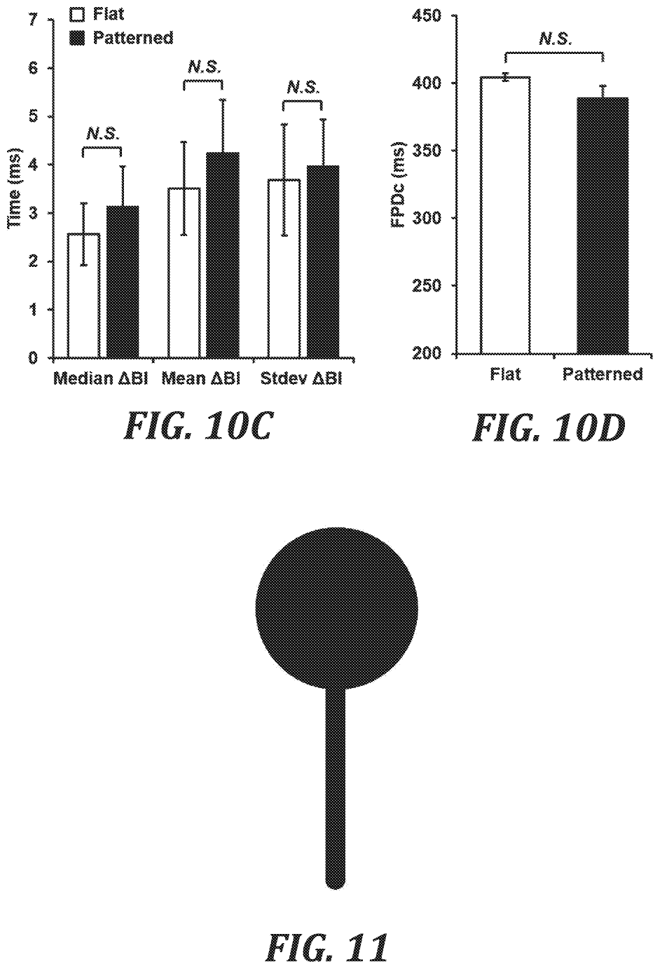

Next, an investigation was carried out to determine whether the observed changes in hPSC-CM phenotype correlated with any physiological or maladaptive changes in electrophysiological function in these cells. FIGS. 10A-10D shows a comparative study of baseline electrophysiology in flat and patterned hPSC-CM cultures. FIG. 10A shows a representative baseline field potential recording from hPSC-CM monolayers maintained on flat Nafion MEAs for 21 days. FIG. 10B shows a representative baseline field potential recording from hPSC-CM monolayers maintained on patterned Nafion MEAs for 21 days. FIG. 10C shows a comparison of beat interval variability metrics calculated from analysis of hPSC-CMs maintained on flat and patterned MEAs for 21 days. Median difference in beat interval (ΔBI), mean ΔBI, and the standard deviation of the ΔBI were compared in order to provide quantification of baseline arrhythmic properties in line with previously published methods. FIG. 10D shows field potential durations (corrected for beat rate; FPDc) recorded from hPSC-CM monolayers maintained on flat and patterned MEAs for 21 days. This study showed that spontaneous baseline beat rate was not found to differ significantly between unpatterned and patterned cells following 21 days in vitro. Critically, analysis of beat-to-beat-variance (through comparison of the median and mean difference in beat interval period from one beat to the next, as shown in FIGS. 10A, 10B, and 10C, highlighted that both unpatterned and patterned cells exhibited regular and consistent beat intervals at baseline, thereby confirming the presence of stable, non-arrhythmic populations for subsequent assessment. Analysis of spontaneously evoked field potential durations (corrected for beat rate using Fridericia's formula; FPDc) illustrated that both flat and nanotopographically-patterned hPSC-CM monolayers exhibited statistically similar FPDc after 21 days in vitro, as shown in FIG. 10D. FPDc correlates with QT interval in vivo, and the data presented here suggest that hPSC-CMs from CDI are able to generate QT intervals that closely recapitulate those observed in the native human adult myocardium (˜400 ms). Consistent FPDc, beat rate, and inter-beat interval in flat and patterned cells suggests that provision of nanotopographic cues to cultured hPSC-CM monolayers had no adverse effect on field potential waveform or pacing in these cells.

Additionally, interrogation of monolayer culture by MEA facilitates assessment of conduction velocity in a manner that is difficult when using single cell analysis methods, such as patch clamp electrophysiology. For all propagation experiments discussed herein, longitudinal conduction dictates the vertical orientation on the MEA as all nanopatterns were oriented to run vertically during pattern generation. FIG. 6I depicts measurement of conduction velocity (CV) across hPSC-CM monolayers on flat and nanotopographically-patterned MEAs. Conduction was measured specifically in both the transverse (TCV) and longitudinal (LCV) orientations and underlying nanotopography was organized to run longitudinally. In all presented data, *p<0.03, **p<0.003, ***p<0.001. As shown in FIG. 6I, conduction velocities measured from hPSC-CMs on nanoMEA devices of the present disclosure were found to be highly anisotropic, thereby more closely mirroring the directed propagation patterns present in the native myocardium. Furthermore, longitudinal conduction velocities measured from patterned cells were substantially faster than similar measurements taken from flat controls, as well as global (non-directional) conduction velocity measurements. Specifically, longitudinal conduction velocity measured from patterned cells was recorded as 32.73 cm/s±9.51, whereas flat controls produced longitudinal and global conduction speeds of 10.32 cm/s±2.62 and 23.49±4.57, respectively.

### NanoMEAs Increase Cardiomyocyte Sensitivity to Cardiotoxic Compounds that Target Structural Features of the Cell

Given the observed improvements in structural and functional maturation of hPSC-CMS cultured in nanoMEA assays, a variety of experiments were carried out to establish whether observed differences in cardiomyocyte structural and functional phenotypes led to alterations in cellular responses to cardiotoxic agent exposure (e.g., one example of a pharmacologically active compound). Specifically, a study was done to analyze whether unpatterned and nanotopographically-patterned cells exhibited different sensitivities to compounds with arrhythmogenic or conduction blocking activity. The ability to pattern individual wells within a multiwell MEA plate enabled assessment of multiple doses across both surface conditions within a single device, thereby increasing throughput for such comparative studies. Verapamil was first examined as a negative control compound.

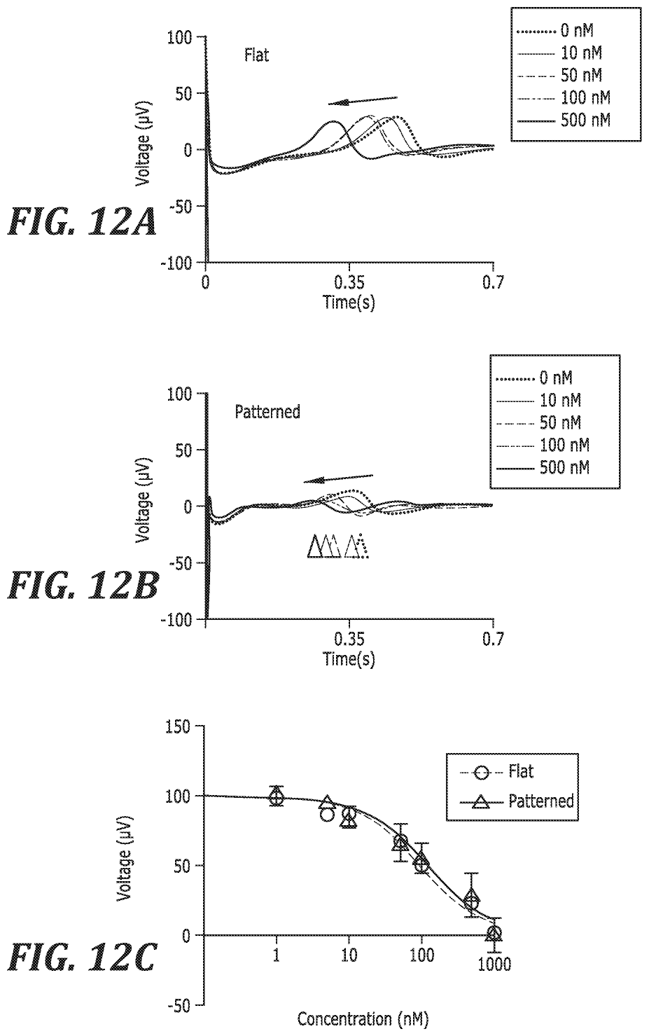

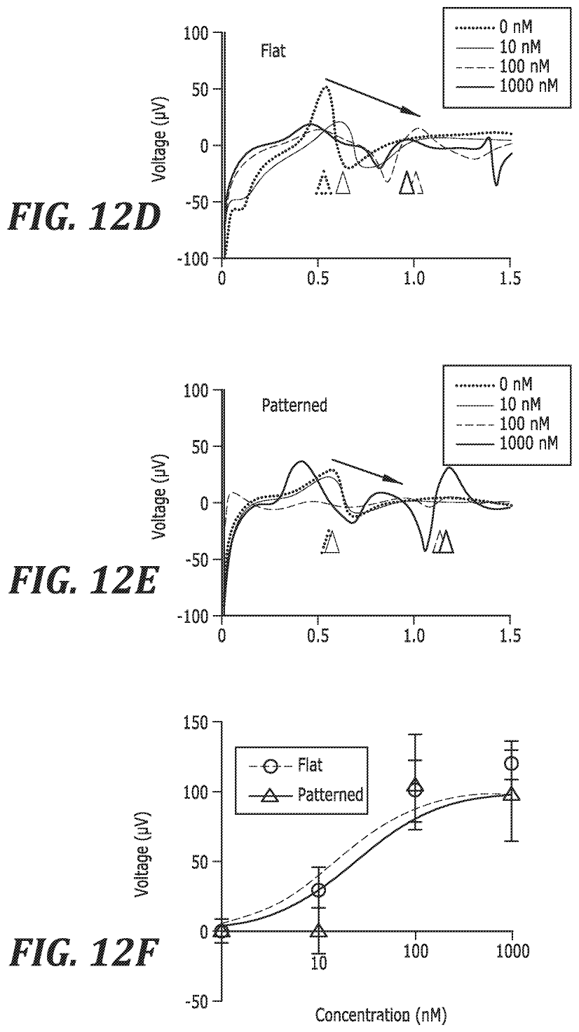

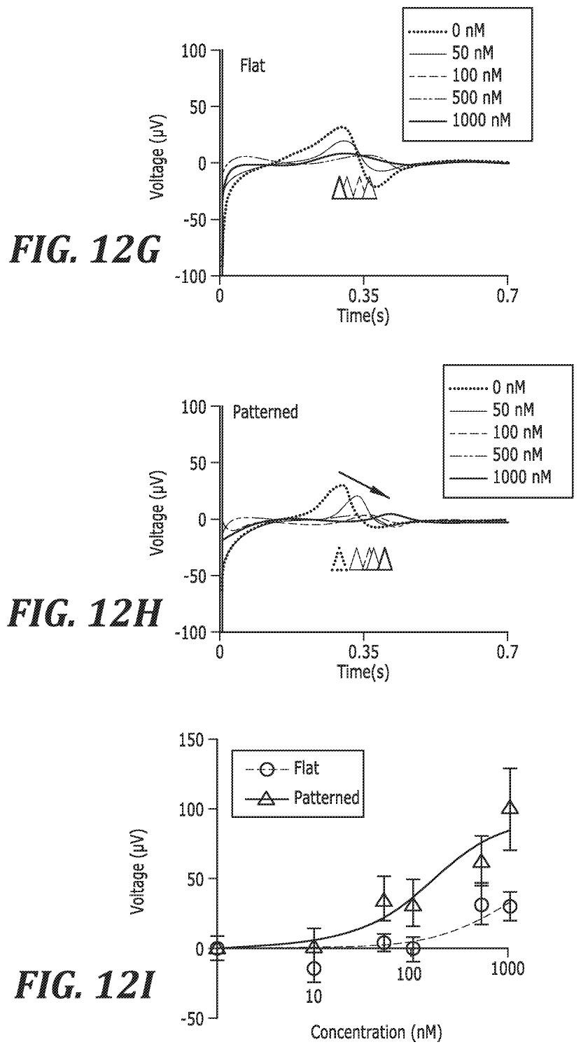

FIG. 12 shows electrophysiological response of flat and patterned hPSC-CMs to treatment with known arrhythmogenic compounds. FIG. 12A depicts representative traces (averaged across 10 beats) recorded from hPSC-CM monolayers on flat MEAs and subjected to increasing doses of verapamil. FIG. 12B depicts representative traces recorded from hPSC-CM monolayers on nanoMEAs and subjected to increasing doses of verapamil. FIG. 12C depicts normalized dose response curve illustrating the effect for increasing concentrations of verapamil on the field potential durations corrected for beat rate (FPDc) of unpatterned and patterned hPSC-CMs. The R² values for the unpatterned and patterned cultures were 0.86 and 0.60, respectively. As has been reported previously, exposure of unpatterned and patterned cells to increasing doses of verapamil produced a significant shortening of FPDc and no notable arrhythmogenic effect. No significant difference was observed in the response of unpatterned and patterned cells.

Cisapride is known to be potent inhibitor of Kᵥ 11.1 (hERG) channel activity in cardiac cells, and capable of causing dangerous prolongation of the QT interval, as well as potential clinical arrhythmias, in patients. Treating cultured hPSC-CMs with cisapride has been shown previously to elicit field potential/action potential prolongation and arrhythmogenic behavior. In line with previously published data, treatment of both unpatterned and nanotopographically-patterned cells with increasing doses of cisapride led to a significant prolongation of the field potential. FIG. 12D depicts representative traces recorded from hPSC-CM monolayers on flat MEAs and subjected to increasing doses of cisapride. FIG. 12G depicts representative traces recorded from hPSC-CM monolayers on nanoMEAs and subjected to increasing doses of cisapride. FIG. 12F depicts normalized dose response curve illustrating the effect of increasing concentrations of cisapride on the FPDc of unpatterned and patterned hPSC-CMs. The R² values for the unpatterned and patterned cultures were 0.51 and 0.46, respectively. This study further showed that supra-physiological doses of the drug (100 nM and 1 μM) were found to induce severe arrhythmogenic behavior and the emergence of early afterdepolarizations and ectopic beats in all unpatterned and patterned cultures examined. At the highest dose examined (1 μM), cisapride was found to elicit a ˜50% FPDc prolongation in cells maintained on both substrates. Specifically, FPDc increased from 531.78 ms±7.99 and 502.60 ms±42.69 on flat and patterned surfaces, respectively, to 690.85 ms±14.89 and 707.06 ms±83.40. No significant difference in the response of cells maintained on different substrates was observed.

Bepridil is a Ca²⁺ release antagonist originally developed to treat angina that has been shown to prolong QT interval in the majority of patients for whom it has been prescribed. The mechanism by which bepridil prolongs QT is yet to be fully elucidated. However, in addition to regulating Ca²⁺ release, it is known that bepridil competes with cTnI for troponin C binding sites, thereby altering Ca²⁺ sensitivity in exposed cells. This suggests that the ability for bepridil to alter cardiomyocyte functional performance may be tied to the presence, organization, and/or function of troponin in these cells. Critically for drug development applications, previous studies with bepridil using conventional MEAs have demonstrated that cultured hPSC-CMs exhibit little electrophysiological response to the compound, even at supra-physiological doses. However, treatment of cardiomyocytes maintained on nanoMEA devices of the present disclosure with bepridil led to a significant prolongation of FPDc in patterned cultures, whereas the change in FPDc measured in bepridil-treated flat controls was not significantly different from baseline (FIGS. 12G, 12H and 12I). FIG. 12G depicts representative traces recorded from hPSC-CM monolayers on flat MEAs and subjected to increasing doses of bepridil. FIG. 12H depicts representative traces recorded from hPSC-CM monolayers on nanoMEAs and subjected to increasing doses of bepridil. FIG. 12I depicts normalized dose response curve illustrating effect of increasing concentrations of bepridil on the FPDc of unpatterned and patterned hPSC-CMs. The R² values for the unpatterned and patterned cultures were 0.49 and 0.76, respectively. This study showed that 1 μM bepridil treatment on flat cells promoted a change in FPDc from 404.46 ms±26.44 at baseline to 434.61 ms±2.14 (a change of just 7% from baseline), suggesting the drug had little impact on the electrophysiology of cardiomyocytes maintained on conventional culture surfaces. Conversely, bepridil treatment on patterned cells was found to increase FPDc from 388.58 ms±13.70 at baseline to 463.72 ms±2.56 at 1 μM bepridil; an increase of roughly 20%.

Carbenoxolone is a gap junction blocker (currently unavailable in the US) that is typically used to treat ulceration of the gastrointestinal tract. In cardiac tissue, it is known to exert a strong conduction blocking effect, inhibiting action potential propagation and in turn affecting synchronous contraction of the tissue. Analysis of cardiac conduction patterns on nanoMEA platform of the present disclosure highlighted that treatment of cells with carbenoxolone significantly reduced conduction speeds in vitro. However, substantial differences were observed in the inhibitory effect of the compound, depending on the substrate condition and the orientation of propagation examined.

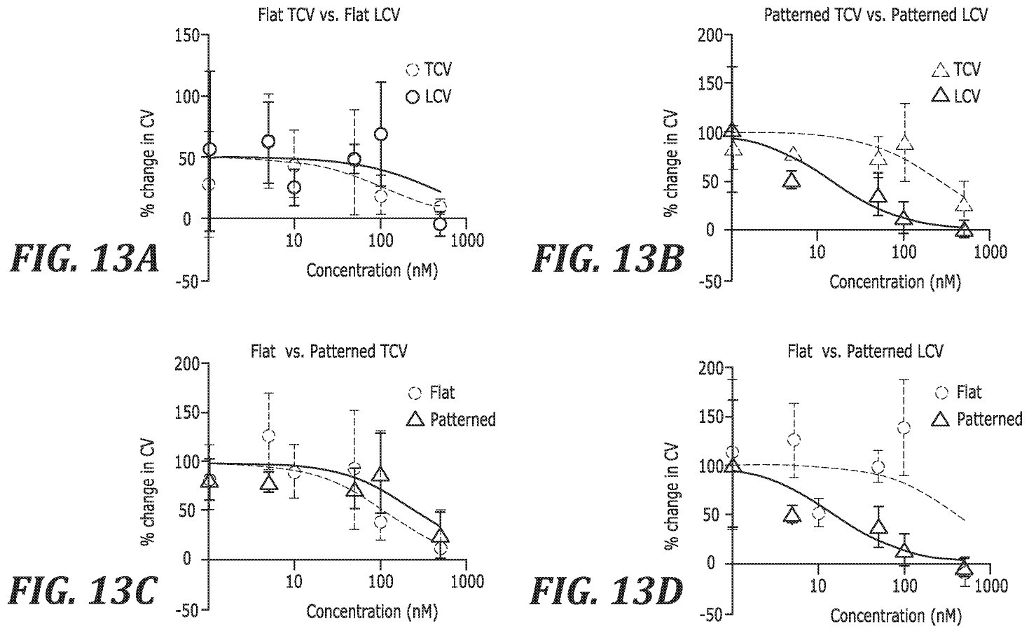

FIGS. 13A-13D show various responses of flat and patterned hPSC-CMs to treatment with the conduction-blocking compound carbenoxolone. FIG. 13A depicts normalized dose response curves illustrating the effect of increasing concentrations of carbenoxolone on the conduction velocity of unpatterned hPSC-CM monolayers. Dose response curves were calculated from analysis of propagation speeds in both the transverse (TCV) and longitudinal (LCV) directions. The R² values for TCV and LCV curve fits were 0.23 and 0.11, respectively. FIG. 13B depicts normalized dose response curves illustrating the effect of increasing concentrations of carbenoxolone on the TCV and LCV of patterned hPSC-CM monolayers. The R² values for TCV and LCV curve fits were 0.21 and 0.28, respectively. FIG. 13C depicts normalized dose response curves illustrating the effect of increasing concentrations of carbenoxolone on the TCV of flat and patterned hPSC-CMs. FIG. 13D depicts normalized dose response curves illustrating the effect of increasing concentrations of carbenoxolone on the LCV of flat and patterned hPSC-CM monolayers.

On flat surfaces, as shown in FIG. 13A, no significant difference was observed in the dose response curves generated for transverse conduction velocity (TCV) and longitudinal conduction velocity (LCV). Furthermore, the inconsistency of the propagation waves on these surfaces led to substantial variability in measured values across experimental repeats, making it difficult to construct well-fitted dose response curves. In comparison, as shown in FIG. 13B, TCV and LCV recorded from nanotopographically-patterned cells exhibited significant differences in response to carbenoxolone treatment, with longitudinal propagation affected at significantly lower doses than transverse propagation. Additionally, the consistency of the propagation waves in these cultures created more reproducible data, tighter error bars, and better fitted dose response curves. The standard deviation of the residuals for the fitted data were 55.25 and 74.19 for flat TCV and LCV, respectively, whereas TCV and LCV curves generated for the patterned data had standard deviation of the residuals of 42.10 and 25.15, respectively. Residual values indicate the vertical distance (in Y units; in this case % change in conduction velocity) of the measured points from the fit line. Accordingly, the reduction in standard deviation of the residuals for the patterned data (compared with flat controls) suggests the data points fall closer to the fit line and indicate an overall improvement in the fit. As shown in FIG. 13C, comparison of TCV values from flat and patterned data sets revealed no significant difference in carbenoxolone dose response. However, as shown in FIG. 13D, analysis of LCV curves from both groups showed increased sensitivity in patterned cells versus flat controls. Specifically, the carbenoxolone IC₅₀s, obtained from flat and nanotopographically-patterned LCV data, were calculated as 398.1 nM and 14.71 nM, respectively.

### NanoMEAs Exacerbate Electrophysiological Defects in Hypertrophic Cardiomyopathy Patient-Derived hPSC-CMs

Hypertrophic cardiomyopathy (HCM) manifests as a thickening of the myocardial tissue, typically due to mutations in genes encoding specific sarcomeric proteins. In patients, these structural defects lead to impairment of electrophysiological function and severe arrhythmogenic behavior. Given the impact of cardiomyocyte structural defects on myocardial electrophysiological function in vivo, and the impact of nanotopographic substrates on cardiomyocyte structure discussed above, an investigation was carried out to determine whether hPSC-CMs derived from an HCM patient (MYH7 R663H variant) exhibited any notable differences in field potential properties when grown on nanoMEA assay plates.

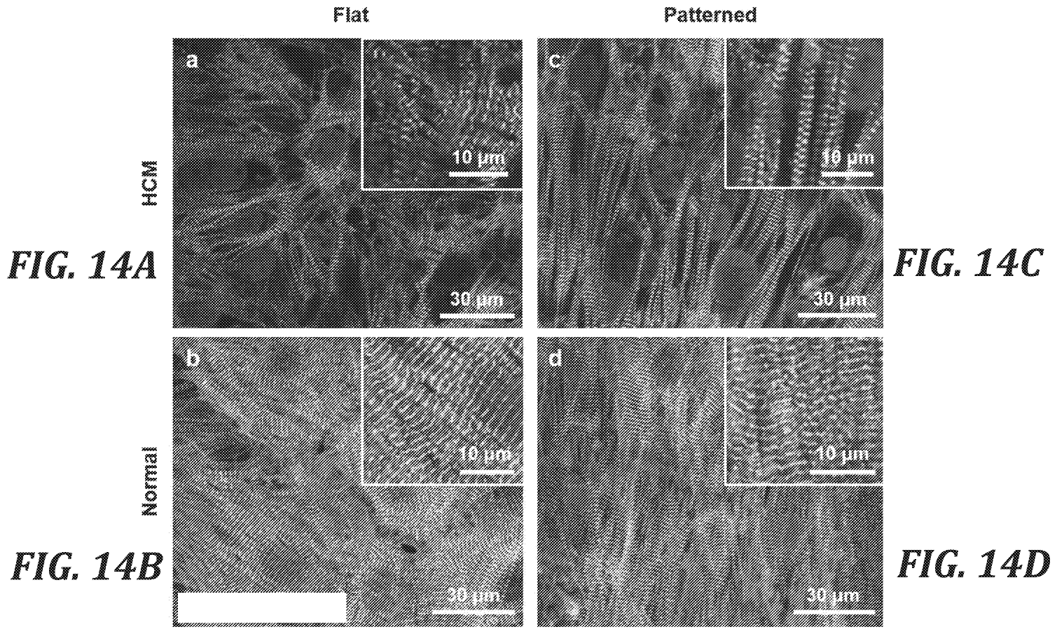

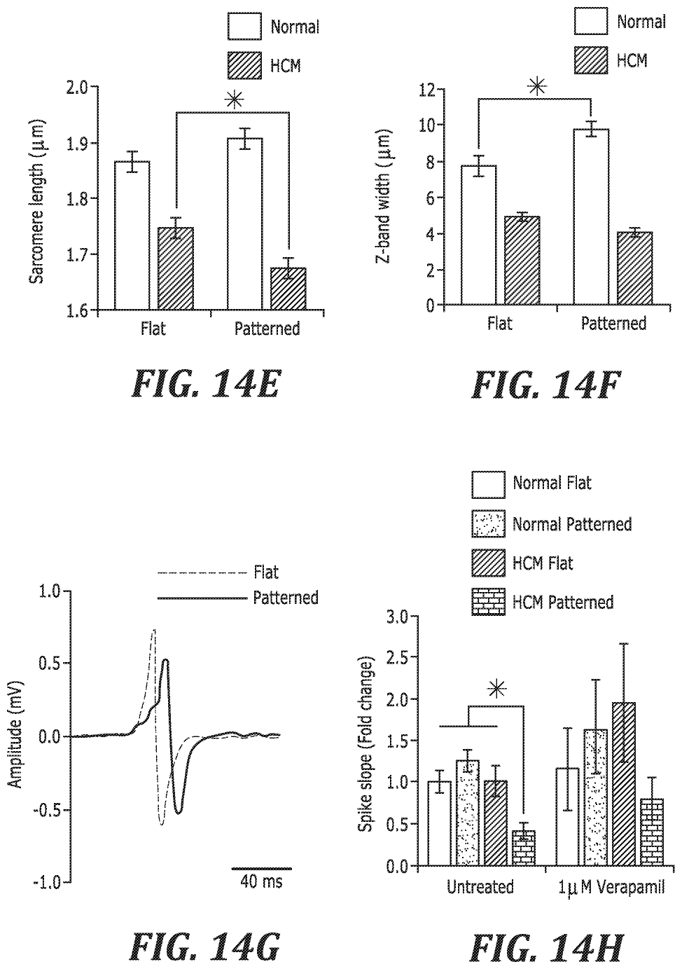

FIG. 14A-14H shows various structural and electrophysiological responses of normal and HCM hPSC-CMs to culture on nanoMEA assay platform of the present disclosure. FIG. 14A depicts an immunostained image of hPSC-CMs obtained from HCM patients and maintained on flat Nafion substrates. FIG. 14B depicts an immunostained image of hPSC-CMs obtained from an unaffected family member related to the examined HCM patient and maintained on flat Nafion substrates. FIG. 14C depicts an immunostained image of hPSC-CMs obtained from HCM patients and maintained on patterned Nafion substrates. FIG. 14D depicts an immunostained image of hPSC-CMs obtained from an unaffected family member related to the examined HCM patient and maintained on patterned Nafion substrates. FIG. 14E depicts quantification of sarcomere length measured from HCM and familial control hPSC-CMs maintained on flat and patterned Nafion substrates. FIG. 14F depicts quantification of z-band width measured from HCM and familial control hPSC-CMs maintained on flat and patterned Nafion substrates. FIG. 14G depicts representative QRS complex traces collected from HCM hPSC-CMs on flat and patterned MEAs. FIG. 14H depicts fold-change in average spike slope (Q-R angle) measured across 30 beats from normal and HCM hPSC-CMs on flat and patterned MEAs. Measurements were collected from untreated cells and cells exposed to 1 μM verapamil for 30 minutes. In all presented data, *p<0.05.

Immunocytochemical analysis of sarcomere development in cultured HCM cardiomyocytes in FIGS. 14A-14F highlighted that nanotopographically patterned HCM cells develop significantly shorter sarcomeres and smaller z-band widths than equivalent cells on flat surfaces, suggesting that provision of nanotopographic structural cues exacerbates the physical phenotype within this cell model of HCM. Despite the structural breakdown observed in patterned HCM cells, no increase in arrhythmia was observed in cells maintained on nanoMEAs compared with those grown on conventional MEAs. However, a prolongation of the depolarizing spike width was observed after just 7 days in vitro, as shown in FIG. 14G and FIG. 14H. No difference was observed in normal control cells on different substrates, suggesting that the phenotypic difference recorded in HCM cells may have resulted from a disease-specific response to culture on nanotopographic substrates. A trend towards smaller field potential amplitudes was also observed between unpatterned and patterned HCM cells, but this difference was not significant. Verapamil is often used to treat HCM, and so was examined as a means to reverse the observed differences in depolarizing spike width between patterned and unpatterned cells. Treatment with 1 μM verapamil improved patterned HCM spike slopes to the point where no significant difference was observed between any experimental groups.

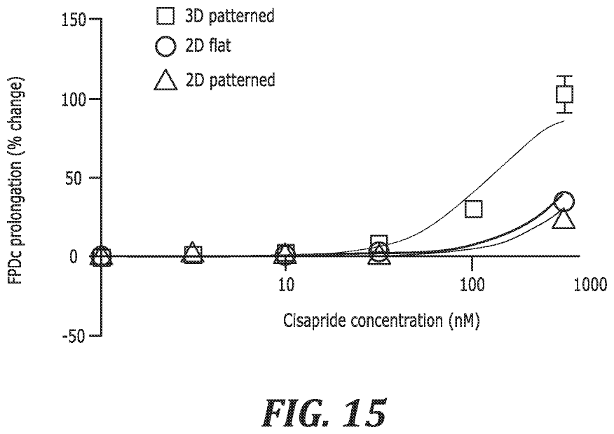

The utility of the nanoMEA platform of the present disclosure is not limited to the cells developed in 2D monolayers. Multiple layers of cardiac cells forming 3D cardiac cultures can be maintained on a nanoMEA device of the present disclosure for more accurate or sensitive screening for preclinical drug screening. FIG. 15 depicts engineered cardiac tissue responses to treatment with increasing doses of the hERG channel blocker cisapride on 2D unpatterned, 2D patterned and 3D patterned cell cultures. FIG. 15 shows that 3D cardiac tissues maintained on the nanoMEA platform exhibit significantly greater responses to treatment with the compound than either flat (unpatterned) or patterned 2D counterparts. In this experiment, cells are first plated onto a nanoMEA substrate in a 4 μL droplet of medium. 17,500 cells are resuspended in this droplet and allowed to adhere to the substrate for 1 hour. At this point, a further 17,500 cells in 2 μL of medium are plated on top of the existing droplet and allowed to adhere to the first layer of cells. This process is repeated 2 more times to form 4 layers of cells on top of the patterned substrate. Cells on the first layer align in parallel with the underlying substrate and subsequent layers align in parallel with the layer below. Cardiomyocytes are mixed with cardiac fibroblasts at a ratio of 10 cardiomyocytes to 1 fibroblast. An hour after the last plating, the MEA wells are flooded with additional medium to maintain the cells and the constructs are then cultured out to 21 days before being used for drug screening assessment. An assessment with the hERG blocker cisapride shows significantly stronger reactions to treatment with the compound, in terms of field potential duration prolongation; a major indicator of arrhythmogenic potential. These data suggest the 3D cardiac cultures maintained on nanoMEAs can offer a more sensitive screening platform for predictive preclinical drug screening.

hPSC-CMs are seen by many as a promising assay for improving the accuracy of preclinical drug development protocols. However, their inability to produce adult-like structural and functional properties has raised questions as to whether these human cells are capable of providing preclinical data that is more meaningful than what is currently possible with existing methodologies. In some embodiment, the primary objectives of the present disclosure are to establish robust, high-fidelity, nanotopographically-patterned microelectrode arrays and to validate these engineered devices in terms of their capacity to enhance cardiomyocyte structure, electrophysiological function, and response to proarrhythmogenic compound exposure.

Human myocardial tissue possesses a complex structural hierarchy on multiple length scales ranging from macroscopic, tissue-level organization to subcellular nanoscale guidance cues. Based on this understanding of the myocardial structural niche, the present disclosure demonstrates how provision of biomimetic, nanoscale substrate cues, mimicking the size and orientation of myocardial extracellular matrix (ECM) fibers, can be used to promote the structural and functional development of cultured human cardiomyocytes. In particular, the experimental data derived from the present disclosure demonstrate how ion-permeable nanotopographic patterns can be utilized to attenuate the structural development of hPSC-CM monolayers and 3D cultures on nanoMEAs.

Previous analysis of ssTnI and cTnI as a quantifiable ratio-metric maturation signature for induced pluripotent stem cell-derived cardiomyocytes has highlighted that such cultured cells express ssTnI consistently throughout long-term (9-month) culture, whereas expression of cTnI increases over this period. The significant upregulation of cTnI protein in cells maintained on nanotopographic patterns suggest that substrate topography was able to promote significant sarcomeric maturation over the 3-week culture period examined. The increase in cTnI expression after 3 weeks in nanotopographically-patterned culture correlates with increased expression in unpatterned cells after 2, 6, and 9 months in vitro, as reported previously. As such, the troponin protein expression data presented here, coupled with the observed improvements in cellular alignment and sarcomeric development, confirm that nanotopographically-patterned Nafion culture substrates promote more rapid maturation of hPSC-CM structure compared with cells maintained on conventional flat surfaces.

Further, the 48-well nanoMEA device of the present disclosure demonstrated the utility of the nanoMEA platform for high-throughput analysis of the electrophysiological properties of hPSC-CM monolayers. The experimental data demonstrated that biophysical regulation of cardiomyocyte structural maturation, via presentation of nanoscale topographic cues, leads to concurrent changes in functional output. Specifically, nanotopographically-patterned Nafion substrates was found to promote the development of conduction patterns and speeds in hPSC-CM monolayers that more accurately recapitulate those found in mature human cardiac tissue. The recorded polarization of gap junction proteins in patterned cell monolayers, coupled with cardiomyocyte alignment and elongation in parallel with the underlying topography, likely account for the anisotropic propagation waves observed. Furthermore, they demonstrate how recapitulation of native myocardial architecture can help cultured cardiac monolayers function in a manner more representative of native myocardial tissue.

Despite the improvements observed in the structural and functional phenotype of patterned hPSC-CMs, the hERG channel blocker, cisapride, was not found to exert an altered electrophysiological response in patterned versus unpatterned hPSC-CM monolayers. Conversely, the calcium channel blocker, bepridil, was found to exert a more powerful FPDc prolongation effect in patterned versus unpatterned cells. The capacity for bepridil to elicit a more pronounced effect on cultured cardiac monolayers may be attributable to its interaction with cytoskeletal elements within cardiomyocytes. It has been demonstrated that bepridil accumulates within cardiac cells and binds tightly to F-actin. More recently, it has been shown that bepridil competes for troponin C binding sites with cTnI, which alters Ca²⁺ sensitivity in exposed cells. The fact that bepridil interacts closely with cytoskeletal elements and contractile proteins, which are shown to be upregulated and reorganized in patterned cardiomyocyte populations, provides an indication as to why this drug elicited altered responses when applied to the cells maintained on nanoMEA devices.

Analysis of transverse and longitudinal conduction velocity (TCV and LCV, respectively) in patterned hPSC-CM monolayers demonstrated that propagation was significantly more anisotropic in patterned cells. Moreover, comparison of results for carbenoxolone-induced changes in the LCV and TCV of flat and patterned monolayers highlighted that patterned cells exhibit increased, and more consistent, sensitivity to the conduction-blocking compound. The fact that Cx43 was more highly expressed in patterned cells, and localized preferentially at cardiomyocytes' polar ends, may explain the altered ability to detect carbenoxolone effects when examining propagation speeds in flat and patterned cells and along different orientation planes. The IC₅₀ for carbenoxolone calculated from LCV measurements in patterned cells was found to be 14.71 nM; roughly 27-fold lower than results calculated from unpatterned LCV measurements. Carbenoxolone plasma concentrations in patients vary substantially, with numbers in the 55.06 μM±21.76 range. However, clinically relevant unbound concentrations are considerably lower; and have been reported in the low nM range. The fact that the carbenoxolone IC₅₀ calculated from flat LCV data was measured at 398.1 nM, suggests that flat hPSC-CM monolayers require higher than physiological doses of the drug before an effect can be reliable detected. Critically, the experimental results derived from this disclosure indicate that structural organization of the cardiac monolayer, that enables study of longitudinal propagation specifically, facilitates detection of compound action at lower doses, thereby offering a more physiologically accurate prediction of carbenoxolone activity.

Collectively, the bepridil and carbenoxolone data presented herein demonstrate that the nanoMEA platform of the present disclosure improves the predictive capacity of current MEA-based preclinical screening paradigms in terms of their ability to model human myocardial responses to drug exposure. However, the reported cisapride data suggests that the capacity for matured cells to respond to compound treatment depends heavily on the mode of action of the compound being tested and the specific maturation cues provided to the cells. Given the complexity of the cardiac niche in vivo, it is unlikely that any one maturation cue will be capable of bringing about the development of a phenotype in cultured cardiac cells that elicits clinically representative responses to all classes of compounds. The investigation of combinatorial maturation strategies could therefore be considered as a means to further enhance the predictive capacity of cultured hPSC-CMs and thereby improve their utility in preclinical compound efficacy and toxicity studies. This does not, however, limits the use of nanoMEAs for wide ranging preclinical evaluations; rather, this technology should be considered a baseline platform from which to build more complex cardiac maturation methodologies. In such instances, the nanoMEA platform could offer an effective base screening tool with which to evaluate additional maturation stimuli, such as thyroid hormone or miRNA cocktails, in terms of their capacity to further advance cardiomyocyte structural and functional development in vitro.

The development of induced pluripotent stem cell technology has facilitated the establishment of cell lines carrying patient-specific mutations for comprehensive in vitro analysis. Given the importance of cardiomyocyte structure in facilitating function, and the effect of our nanotopographies on cardiomyocyte structural phenotype, the nanoMEA platform of the present disclosure was used to determine whether hPSC-CMs carrying a mutation in a cytoskeletal-related protein would exhibit downstream functional defects that were more apparent when cells were cultured on nanotopographically patterned surfaces. To that end, hypertrophic cardiomyopathy (HCM) patient-derived hPSC-CMs were established on both flat and patterned MEAs for electrophysiological assessment. By day 7, stratification of phenotype between unpatterned and patterned cells had emerged, with patterned cells exhibiting significant prolongation of the depolarizing spike compared with unpatterned controls. No significant difference was observed in normal cells on different substrates, suggesting that the provision of structural cues to HCM cells in vitro may help to amplify their disease phenotype, likely through promoting sarcomeric development and thereby exacerbating the structural defect present in these cells. This hypothesis was supported by immunocytochemical data, which highlighted that sarcomere lengths and z-band widths were shorter in HCM cells maintained on nanotopographically patterned substrates than those on flat surfaces. Previously published data in the prior art has demonstrated that electrocardiograms (ECGs) from HCM patients exhibit significant QRS prolongation in vivo. Critically, Q wave abnormalities have been reported in HCM patients carrying myosin heavy chain mutations. Concordance of the nanoMEA data with such patient results suggests provision of physiologically relevant matrix cues to overlying cells may help to promote the development of more clinically relevant phenotypes for further mechanistic and therapeutic study. The ability for verapamil treatment to partially restore normal waveform features in HCM cells further highlights the potential utility of the nanoMEA as a platform for assessing the abilities of novel compounds to ameliorate disease-specific electrophysiological defects.

Nanotopographically patterned Nafion substrates exert a strong influence on hPSC-CM monolayer structural development. Use of the novel nanoMEA device of the present disclosure confirmed that these structural changes correlate with altered electrical propagation patterns that mirror conduction properties in the human myocardium. Improved structural development enhanced hPSC-CM sensitivity to known cardiotoxic compounds that interact with structural elements within the cell. In addition, analysis of HCM cells demonstrated how the nanoMEA could help stratify disease-specific electrophysiological defects in structural cardiomyopathic conditions. Thus, the nanoMEA of the present disclosure represents an exciting new tool for studying structure-function interplay in human cardiac tissue and will be useful in improving current preclinical screening modalities to accelerate drug development.

As shown above through numerous examples of embodiments of the present disclosure, the use of the ion-permeable polymer Nafion to fabricate the described nanotopographic features constitutes a simple, cost-effective, and reproducible means to organize and enhance hPSC-CM development in vitro. The nanoporous nature of the Nafion polymer facilitates efficient signal exchange between underlying electrodes and overlying cells, enabling effective analysis of field potential properties in cardiac cultures. The fabrication process does not require clean-room access and can be adapted to substrates of various length scales as required by a desired application for various types of cells or tissues. As such, the described embodiments of the present disclosure constitute a versatile tool to assay the electrophysiology of any electrically active cell type, whether human or non-human, including cardiomyocytes, skeletal muscle cells, cortical neurons, motor neurons, and sensory neurons. Nanotopography has been shown to enhance or influence the structural development of many cell types, including cardiomyocytes, skeletal muscle cells, smooth muscle cells, endothelial cells, human embryonic stem cells, induced pluripotent stem cells, mesenchymal stem cells, fibroblasts, epithelial cells, and cancer cells. These cells interact with the substrate nanotopography and develop unique maturation characteristics based on such nanotopographic cues. Thus, the nanoMEA platform of the present disclosure can be used to assay the electrophysiology of any of these types of cells with useful readouts of the changes in functional phenotype and other maturation characteristics of those cell types.

Additionally, due to its ability to detect or measure electrophysiological activity in spatially and/or directionally controlled manner with greater sensitivity, the nanoMEA platform of the present disclosure can be used or adapted to measure directional changes in electrophysiological activity. The nanoMEA platform can be also used or adapted to detect or measure extracellular network activity of cells in a spatially or directionally controlled manner, providing a useful tool for screening or characterizing other types of cells such as neuron cells.

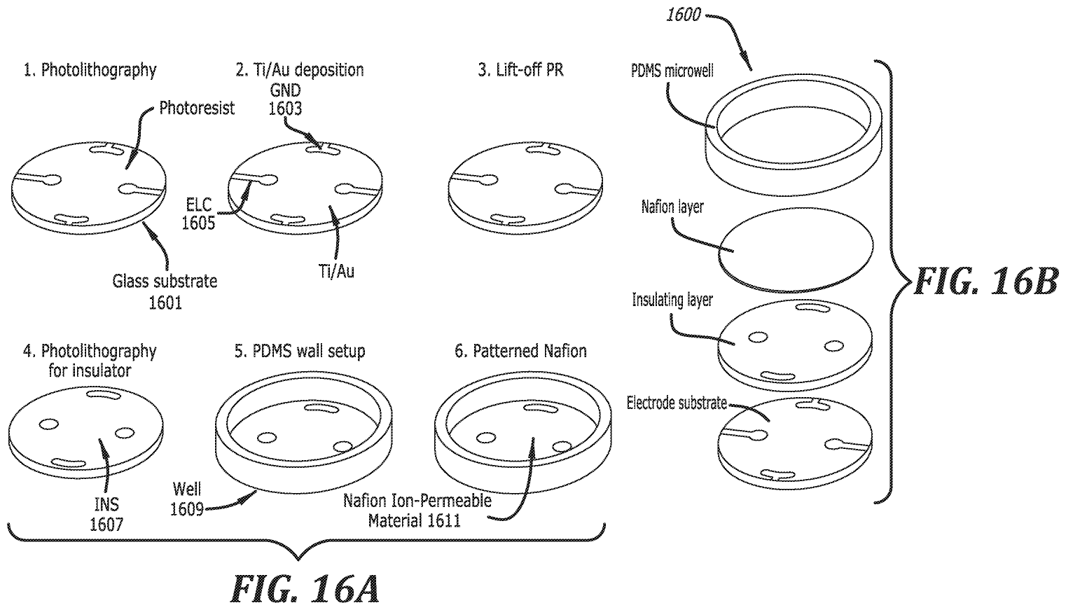

FIG. 16A depicts an example method of device fabrication, and FIG. 16B depicts an exploded view of the complete device architecture 1600, in accordance with an embodiment of the present disclosure. As shown, device 1600 includes substrate 1601, electrodes 1603 (which are coupled to ground), electrodes 1605 (to measure electrical signals from the cells), insulating layer 1607, ion-permeable material 1611 (e.g., Nafion), and culture well 1609.

As illustrated, substrate 1601 (e.g., glass, plastic, or the like) may be coated with photoresist (e.g., a negative or positive resist). A portion of the resist may be exposed to light (e.g., UV light or the like) to define the shape of a plurality of electrodes (including electrodes 1603, and electrodes 1605). Ti may be deposited on the surface of substrate 1610 as an adhesion layer, and then Au may be deposited on top to complete the stack of materials that form the plurality electrodes. However, in some embodiments, other or additional metals, alloys, and compounds (e.g., Ag, doped P+ or N− Si, Ti, Ni, Cu, or the like) may be used to form the plurality of electrodes. After forming electrodes, the photoresist is then removed via solvents like acetone or the like. Another photolithography step is then performed in order to deposit the insulator material 1607 (e.g., another polymer or the photoresist itself) over the entire surface of the substrate and to cover traces from the plurality of electrodes (and prevent shorting between individual electrodes in the plurality of electrodes). In the following steps, a culture well 1609 is created from PDMS, and the ion-permeable material 1611 (e.g, Nafion or the other materials described above) is formed. As shown, culture well 1609 includes an interior cavity having a bottom area, and the bottom area includes the ion-permeable material 1611.

Although not shown in FIG. 16A (but depicted in other figures, and described above), ion-permeable material 1611 includes a textured surface having substantially parallel grooves and ridges. Formation of the textured surface, as previously discussed and shown, may be achieved via PDMS stamping of the grooves and ridges or other techniques. As demonstrated by the experimental results above, is desirable to have the patterned ion-permeable material 1611 disposed between the electrodes and the cells so the cells are not contacting the electrodes. This is because, as detailed above, without ion-permeable material 1611, cell growth may be inhibited or development suboptimal when the cells are in contact with inorganic electrodes. Moreover, as demonstrated by the experimental results presented here, ion-permeable material 1611 does not attenuate the electrical signal transmitted from the cells to the electrodes. Thus the apparatuses, systems, and methods presented here result in both improved cell growth and good electrical characteristics, which could not previously be realized with exposed electrode configurations. Additionally, in some embodiments, the addition of a conductive polymer (e.g., PEDOT) between the electrodes and the ion-permeable material 1611 may further reduce electrical signal noise.

As depicted in the exploded view of device 1600, textured surface is on a first side of the ion-permeable material 1611, and substrate 1601 is disposed proximate to a second side of ion-permeable material 1611, opposite the first side. As depicted, the plurality of electrodes disposed between substrate 1601 and ion-permeable material 1611, are positioned to measure an electrical signal that passes through ion-permeable material 1611 (e.g., to electrically interact with cells disposed in the substantially parallel grooves, when cells are disposed in the substantially parallel grooves). Ions may move through nanopores in ion-permeable material 1611 to permit the electrical interaction between the electrodes and the cells. Specifically, Nafion is a fluoropolymer-copolymer with sulfonate groups and protons on the SO₃H (sulfonic acid) groups “hop” from one acid site to another. However, one of skill in the art will appreciate that other ion-permeable materials may employ other mechanisms to move ions in the bulk of the material and create an electric field. Although omitted from FIG. 16B but depicted elsewhere, in some embodiments a conductive polymer (e.g., PEDOT or the like) is disposed between the plurality of electrodes and ion-permeable material 1611. As shown, insulator material 1607 is disposed (vertically) between substrate 1601 and ion-permeable material 1611, and disposed (laterally) between the individual electrodes in the plurality of electrodes. It is appreciated that in the depicted embodiment, the electrodes are fully covered by ion-permeable material 1611 and would not be in physical contact with cells disposed on ion-permeable material 1611.

As shown in other figures (see e.g., FIGS. 2A-2H), the exploded device architecture depicted may be repeated many times across the substrate in order to enable the characterization of many cell cultures at the same time. This ensures both reproducible data and the ability to test many pharmacologically active substances on the cells at the same time.

One of skill in the art will appreciate that the device depicted in FIG. 16B may be part of a larger system (see e.g., FIG. 4D). The system may include a processor having logic (implemented in hardware, software, or a combination of both) that when executed by the system causes the system to perform operations. Operations may include applying a voltage (which may have any voltage or current waveform desired) through ion-permeable material 1611 and cells, and/or measuring an electrical signal through ion-permeable material 1611 from the cells. The processor may also perform any other number of actions described elsewhere in the instant disclosure.

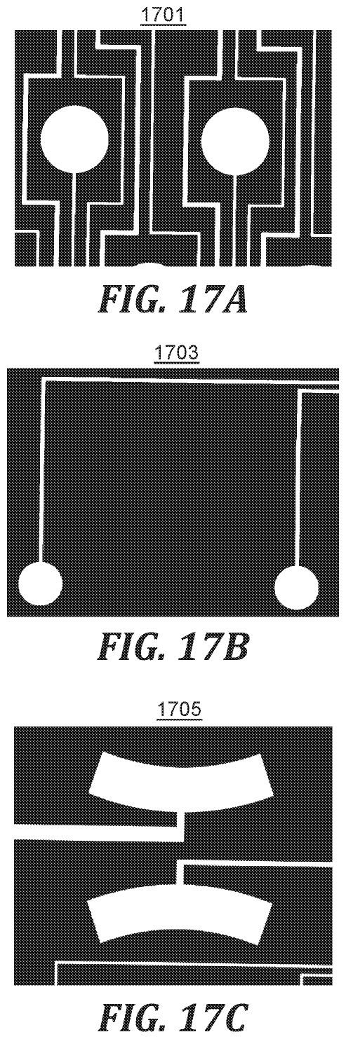

FIGS. 17A-17C depict several electrode shapes, in accordance with an embodiment of the present disclosure. Architecture 1701 in FIG. 17A illustrates contact pads which may be substantially circular and surrounded by metal traces. Similarly, architecture 1703 in FIG. 17B depicts substantially circular electrodes which may be used to receive the electrical signal through the ion-permeable material from the cells. Architecture 1705 in FIG. 17C depicts ground electrodes which (as shown in FIG. 16A) may be disposed laterally between the electrodes used to measure the electrical signal. The ground electrodes may reduce noise in the electrical signal. As shown, the ground electrodes may be substantially crescent shaped, with traces attached to their convex surface. Moreover, in some examples, the ground electrodes may be substantially concentric with the wall of the culture well (see e.g., FIG. 16B) to evenly distribute electric fields in the device. It is appreciated that, one ground electrode may be used while recording electrical signals from the cells, and the other ground electrode may be used while stimulating the cells.

In one embodiment, the width of the lines contacting the electrodes that measure the electrical signals of the cells may be 15 μm, and the diameter of the electrodes themselves may be 30 μm. In another or the same embodiment, the width of the lines contacting the ground electrodes may be 30 μm, while the ground electrodes themselves may measure 1400 μm (from one end of the arc to the other)×300 μm.

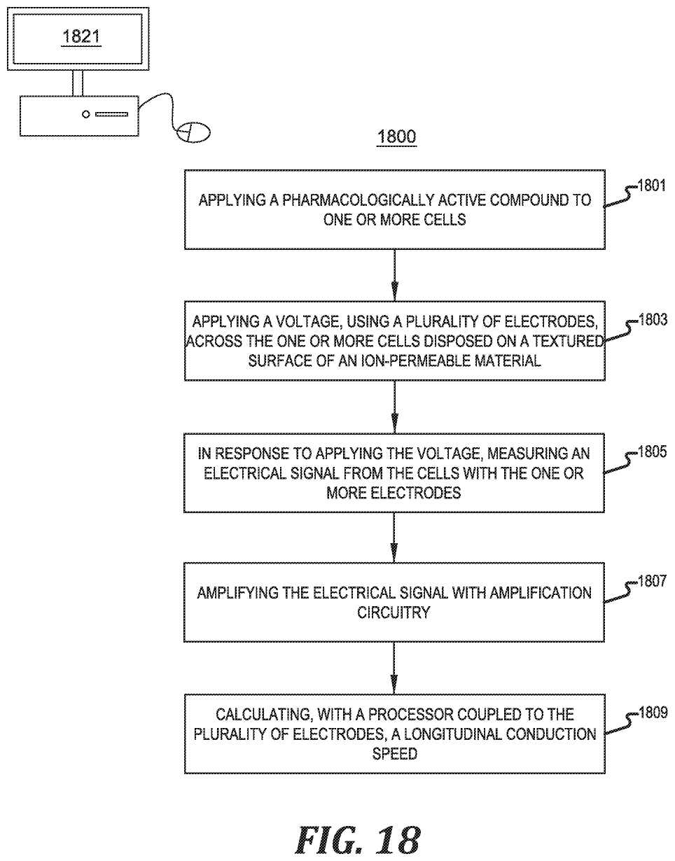

FIG. 18 depicts a method 1800 of measuring the electrical properties of one or more cells, in accordance with the teachings of the present disclosure. It is appreciated that the blocks 1801-1809 may occur in any order an even in parallel. One of ordinary skill in the art will further appreciate that blocks may be added to, or removed from, method 1800 in accordance with the teachings of the present disclosure. Method 1800 may be performed in part by processor 1821 and other hardware components described and depicted elsewhere in the instant disclosure.

Block 1801 shows applying a pharmacologically active compound (e.g., any compound that could induce a change in the structure or behavior of the cells: medications, toxins, new drugs, etc.) to one or more cells (e.g., cardiomyocytes or the like in a 3D tissue culture) disposed on the devices described above (see e.g., FIG. 16B). As shown, this may occur prior to applying a voltage to the cells.

Block 1803 illustrates applying a voltage, using a plurality of electrodes, across the one or more cells. As shown and described above, the cells may be disposed on a textured surface of an ion-permeable material, and the textured surface is on a first side of the ion-permeable material. The plurality of electrodes are disposed proximate to a second side of the ion-permeable material, opposite the first side. The voltage applied to the cells may be used to probe electrical characteristics of the cells.

Block 1805 depicts, in response to applying the voltage, measuring an electrical signal from the cells with the one or more electrodes. In the depicted embodiment, the electrical signal is measured through the ion-permeable material. Put another way, the electrical signal (e.g., electrical signals generated by the cell, a resistance of the cells, a change in conductivity of the cells, or the like) will travel through the ion-permeable material to reach the electrodes. This may be achieved by ions migrating through non-scale pores in the material. In some embodiments, the ion permeable material may include at least one of Nafion, polyethylene terephthalate (“PETG”), track-etched membranes, gelatin, Matrigel, hydrogel, poly-acrylamide, N-isopropylacrylamide (“Poly-NIPAM”), agarose gels, or dextran gels.

In one embodiment, applying the voltage across the one or more cells includes applying a voltage through the ion-permeable material and a conductive polymer disposed between the plurality of electrodes and the ion-permeable material. The conductive polymer may include Poly(3,4-ethylenedioxythiophene) (“PEDOT”) or PEDOT polystyrene sulfonate (“PEDOT:PSS”). It is appreciated that this polymer is “conductive” relative to other polymers, but may be less conductive than inorganic semiconductors and metals. However, conductivity values as high as 10²-10⁴ S/m have been reported. Conductive polymers may have alternating single and double bonds along the polymer backbone (conjugated bonds) or are composed of aromatic rings such as phenylene, naphthalene, anthracene, pyrrole, and thiophene which are connected to one another through carbon-carbon single bonds.

In the depicted embodiment, measuring the electrical signal includes measuring an electrophysiological activity of the one or more cells in one or more directions on the textured surface. In some embodiments, the electrophysiological activity is characterized by increased field potential in electrically active cell types. In other or the same embodiments, the electrophysiological activity is characterized by anisotropic electrical propagation patterns in electrically active cell types.

Block 1807 illustrates amplifying the electrical signal with amplification circuitry (e.g., an operational amplifier) included in the processor and coupled to the plurality of electrodes. As stated elsewhere, the processor may include a variety of software and hardware systems (e.g., “logic”) that may be used to perform a number of calculations such as amplifying the electrical signal received by the electrodes from the cells.

Block 1809 illustrates calculating, with a processor coupled to the plurality of electrodes, a longitudinal conduction speed. It is appreciated that the longitudinal conduction speed may be in the direction of cell alignment, which is in the same direction as the substantially parallel grooves and ridges (which include the one or more cells).

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible machine-readable storage medium includes any mechanism that provides (i.e., stores) information in a non-transitory form accessible by a machine or processor (e.g., a computer, network device, personal digital assistant, manufacturing tool, any device with a set of one or more processors, general-purpose processor configured by firmware/software, programmable gate array, or application specific integrated circuit, etc.). For example, a machine-readable storage medium includes recordable/non-recordable media (e.g., read only memory (ROM), random access memory (RAM), magnetic disk storage media, optical storage media, flash memory devices, etc.). It should be appreciated that a processor broadly includes a processing apparatus (such as the devices described above in this paragraph), memory, and other software or hardware systems. It is also appreciated that a processor may be a distributed system. Logic which may be stored in memory (e.g., RAM, ROM or the like) which may be local or remote.

The above description of illustrated examples of the invention, including what is described in the Abstract, is not intended to be exhaustive or to limit the invention to the precise forms disclosed. While specific examples of the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific examples disclosed in the specification. Rather, the scope of the invention is to be determined entirely by the following claims, which are to be construed in accordance with established doctrines of claim interpretation.

1. An apparatus for biological analysis, comprising:
a substrate;
an ion-permeable material including a textured surface on a first side of the ion-permeable material, and wherein the substrate is disposed proximate to a second side of the ion-permeable material, opposite the first side; and
a plurality of electrodes disposed between the substrate and the ion-permeable material, wherein individual electrodes in the plurality of electrodes are positioned to measure an electrical signal that passes through the ion-permeable material.

2. The apparatus of claim 1, wherein the plurality of electrodes are positioned relative to the ion-permeable material to electrically interact with cells disposed on the textured surface, when the cells are disposed on the textured surface, and wherein the ion permeable material is disposed between the cells and plurality of electrodes so the electrical signal passes through the ion-permeable material from the cells to the plurality of electrodes.

3. (canceled)

4. The apparatus of claim 1, further comprising a conductive polymer disposed between the plurality of electrodes and the ion-permeable material.

5. The apparatus of claim 4, further comprising an insulator material disposed between the substrate and the ion-permeable material, wherein the insulator material is disposed laterally between individual electrodes in the plurality of electrodes to prevent conduction between the individual electrodes.

6. The apparatus of claim 4, wherein the conductive polymer comprises poly(3,4-ethylenedioxythiophene) (“PEDOT”).

7. The apparatus of claim 1, wherein the ion-permeable material is selected from the group consisting of Nafion, polyethylene terephthalate (“PET”), Matrigel, hydrogel, poly-acrylamide, N-isopropylacrylamide (“Poly-NIPAM”), and combinations thereof.

8. The apparatus of claim 1, wherein the textured surface includes substantially parallel grooves and ridges.

9. A system for biological analysis, comprising:
a substrate;
an ion-permeable material including a textured surface shaped to receive cells, wherein the textured surface is on a first side of the ion-permeable material, and wherein the substrate is disposed proximate to a second side of the ion-permeable material, opposite the first side;
a plurality of electrodes disposed between the substrate and the ion-permeable material; and
a processor coupled to the plurality of electrodes, wherein the processor includes logic that when executed by the processor, causes the system to perform operations, including:
measuring an electrical signal through the ion-permeable material with the plurality of electrodes.

10. The system of claim 9, wherein the textured surface includes substantially parallel grooves and ridges.

11. The system of claim 10, wherein measuring an electrical signal with the plurality of electrodes includes measuring an electrophysiological activity of the cells in one or more directions, when the cells are disposed on the textured surface.

12. (canceled)

13. The system of claim 9, further comprising one or more culture wells including an interior cavity having a bottom area, wherein the bottom area includes the ion-permeable material.

14. (canceled)

15. A method of measuring the electrical properties of one or more cells, comprising:
applying a voltage, using a plurality of electrodes, across the one or more cells disposed on a textured surface of an ion-permeable material, wherein the textured surface is on a first side of the ion-permeable material, and wherein the plurality of electrodes are disposed proximate to a second side of the ion-permeable material, opposite the first side; and
in response to applying the voltage, measuring an electrical signal from the cells with the one or more electrodes, wherein the electrical signal is measured through the ion-permeable material.

16. The method of claim 15, wherein measuring the electrical signal includes measuring an electrophysiological activity of the one or more cells in one or more directions on the textured surface.

17. (canceled)

18. The method of claim 16, wherein said electrophysiological activity is characterized by anisotropic electrical propagation patterns in electrically active cell types.

19. The method of claim 15, further comprising:
calculating, with a processor coupled to the plurality of electrodes, a longitudinal conduction speed, wherein the textured surface includes substantially parallel grooves and ridges, and wherein the substantially parallel grooves include the one or more cells, and wherein the longitudinal conduction speed is measured in a direction of the substantially parallel grooves.

20. (canceled)

21. The method of claim 15, wherein the ion-permeable material is selected from the group consisting of Nafion, polyethylene terephthalate (“PET”), Matrigel, hydrogel, poly-acrylamide, N-isopropylacrylamide (“Poly-NIPAM”), and combinations thereof.

22. The method of claim 15, wherein applying the voltage across the one or more cells include applying a voltage through the ion-permeable material and a conductive polymer disposed between the plurality of electrodes and the ion-permeable material.

23. The method of claim 22, wherein the conductive polymer comprises poly(3,4-ethylenedioxythiophene) (“PEDOT”).

24. The method of claim 15, further comprising:
applying a pharmacologically active compound to the one or more cells prior to applying the voltage.

25. The method of claim 24, wherein the one or more cells include cardiomyocytes.

26. (canceled)
